(19) United States
(12) Patent Application Publication
(10) Pub. No.: US 2010/0009007 A1
(43) Pub. Date: Jan. 14, 2010

(54) Non-covalent modification of microparticles and process of preparing same

(75) Inventors: Ramin Darvari, Lexington, MA (US); Julia E. Rashba-Step, Newton, MA (US); Terrence L. Scott, Winchester, MA (US)

Correspondence Address: MARSHALL, GERSTEIN & BORUN (BAXTER), 233 SOUTH WACKER DRIVE, 6300 SEARS TOWER, CHICAGO, IL, 60606-6357, US

(73) Assignees: BAXTER INTERNATIONAL INC., Deerfield, IL (US); BAXTER HEALTHCARE S.A., Wallisellen (CH)

(21) Appl. No.: 12/170,792

(22) Filed: Jul. 10, 2008

(51) Int. Cl. A61K 9/14

(52) U.S. Cl. 424/499

(57) ABSTRACT

The present disclosure is directed to surface-modified microparticles, pharmaceutical compositions thereof, and methods of making and using such particles. The surface-modified microparticles include a microparticle core, and at least one monolayer associated with the microparticle core. The monolayer comprises an amphiphilic polymer or non-ionic polymer grafted to an ionic polymer.

### FIELD OF THE INVENTION

The present disclosure is generally directed to microparticles containing one or more active agents and methods of using such microparticles. More particularly, the present disclosure is directed to microparticles having a modified surface such that the microparticles are capable of altered binding interactions with molecules, cells, and tissues, as compared to microparticles not carrying such surface modifications. The present disclosure is further directed to methods of making and using such surface-modified microparticles.

### BACKGROUND

Microparticles, microspheres, and microcapsules, referred to herein collectively as “microparticles”, are solid or semi-solid particles having a diameter of less than one millimeter, and more typically less than 100 microns, which can be formed of a variety of materials, including proteins, synthetic polymers, polysaccharides, nucleic acids, small molecules, and combinations thereof. Microspheres have been used in many different applications, primarily separations, diagnostics, and drug delivery.

The most well known examples of microparticles used in separations techniques are those which are formed of polymers of either synthetic or protein origin, such as polyacrylamide, hydroxyapatite, or agarose. These polymeric microparticles are commonly used to separate molecules such as proteins based on molecular weight and/or ionic charge, or by interaction with molecules chemically coupled to the microparticles.

In the diagnostic area, spherical beads or particles have been commercially available as a tool for biochemists for many years. For example, microparticles have been derivatized with an enzyme, a substrate for an enzyme, or a labeled antibody, and then interacted with a molecule to be detected, either directly or indirectly. A number of derivatized beads are commercially available with various constituents and sizes.

In the controlled drug delivery area, molecules have been encapsulated within microparticles or incorporated into a matrix to provide controlled release of the molecules. A number of different techniques have been used to make such microparticles from various polymers including phase separation, solvent evaporation, emulsification, and spray drying. Generally, the polymers form the supporting structure of the microparticles, and the drug or molecule of interest is incorporated into the supporting structure. Exemplary polymers used for the formation of microparticles include homopolymers and copolymers of lactic acid and glycolic acid (PLGA), block copolymers, and polyphosphazenes.

Microparticles useful for drug delivery have been modified by providing coatings (also known as monolayers) on or about the microparticles to fine-tune various properties of the microparticles. U.S. Patent Publication No. 2006/0260777 discloses that such coated (or surface-modified) microparticles can demonstrate altered drug release profiles.

Zahr et al., Langmuir, 21, 403-410 (2005) discloses coating dexamethasone nanoparticles with a polyelectrolyte to form a shell about the nanoparticles and then subsequently modifying the shell by covalently attaching poly(ethylene glycol) thereto. The disclosed procedure involves directly modifying the surface coating or shell and thus the drug itself is available to react with the activated poly(ethylene glycol) reagent. This can significantly diminish drug activity or even render a drug entirely inactive. Further, the pharmacokinetic and/or pharmacodynamic profiles of the drug can be negatively affected as a result of modification by the activated poly(ethylene glycol) reagent. Therefore, the disclosed method is not suitable for modifying microparticles comprising active agents, particularly active agents comprising reactive (or modifiable functional) groups such as, for example, small molecules having modifiable functional groups and macromolecules having modifiable functional groups

Accordingly, there is an on-going need for development of microparticles and methods for making same, particularly microparticles that can be adapted for use in delivering drugs of interest.

### SUMMARY

The present disclosure is directed to a surface-modified microparticle that includes a microparticle core and at least one monolayer associated with the microparticle core. The microparticle core can include an active agent. The monolayer of the surface-modified microparticle includes an amphiphilic polymer or a nonionic polymer grafted to an ionic polymer. The degree of grafting of the amphiphilic polymer or nonionic polymer to the ionic polymer typically can be from about 1% to about 30%.

The present disclosure is also directed to a pharmaceutical composition that includes a plurality of such surface-modified microparticles.

The present disclosure is also directed to a method of preparing a surface-modified microparticle that includes providing a microparticle core, admixing an amphiphilic polymer or a nonionic polymer, and an ionic polymer under conditions sufficient to form a grafted polymer, and admixing the grafted polymer and the microparticle core under conditions sufficient to form a surface-modified microparticle. The surface-modified microparticle includes an outermost monolayer, and the outermost monolayer includes the grafted polymer.

### BRIEF DESCRIPTION OF THE DRAWINGS

FIG. 1 is a laser scanning confocal (LSC) micrograph of insulin microparticles coated with a layer of PEG-grafted FITC-labeled polylysine (Example 2). The degree of grafting of PEG to polylysine is (a) about 6.4%, (b) about 12.9%, and (c) about 19.3%.

FIG. 2 is a graph showing the zeta-potential of insulin microparticles coated with a layer of PEG-grafted FITC-labeled polylysine (Example 2). Particles were coated with about 0.15 mg/mL or about 1.5 mg/mL PEG-grafted FITC-polylysine. Zeta-potential is shown for microparticle cores (uncoated), 0% degree of grafting (i.e., having an ungrafted polylysine layer), about 6.4% degree of grafting, about 12.9% degree of grafting, and about 19.3% degree of grafting.

FIG. 3 is a graph showing the zeta-potential of insulin microparticles coated with a layer of PEG-grafted FITC-labeled polylysine (Example 5). Experiment A samples were measured immediately after formation of the surface-modified microparticles. Experiment B samples were stored at 2-8° C. for 15 days after formation of the surface-modified microparticles prior to carrying out the measurements.

FIG. 4 is a graph showing insulin release profiles from microparticles coated with a monolayer of PEG-grafted FITC-labeled polylysine (Example 6). The microparticles were coated with 0 mg/mL (control; no monolayer), about 0.05 mg/mL, about 0.1 mg/mL, about 0.5 mg/mL, or about 1.5 mg/mL PEG-grafted FITC-labeled polylysine.

FIG. 5 is a graph showing the zeta-potential of nucleic acid microparticles, each coated with an innermost layer of polyaspartic acid, and an outermost layer of either FITC-labeled polylysine (Example 8) or PEG-grafted FITC-labeled polylysine (Example 9). The PEG-grafted FITC-labeled polylysine coating has a degree of grafting of about 6.4%. Zeta-potential of uncoated nucleic acid microparticles is also shown.

FIG. 6 is a fluorescence micrograph of nucleic acid microparticles coated with an innermost layer of polyaspartic acid, and an outermost layer of PEG-grafted FITC-labeled polylysine (Example 9). The degree of grafting of PEG to polylysine is about 6.4%.

FIG. 7 is a graph showing uptake by CD11b-positive spleen cells of nucleic acid microparticles, each coated with an innermost layer of polyaspartic acid, and an outermost layer of either FITC-labeled polylysine (PLL) or PEG-grafted FITC-labeled polylysine (PLL-PEG) (Example 10).

FIG. 8 is a schematic diagram showing a surface-modified microparticle having two monolayers. The surface-modified microparticle includes a microparticle core 1, an innermost monolayer comprising an ionic polymer 2, and an outermost monolayer comprising an amphiphilic polymer or a nonionic polymer grafted to an ionic polymer 3.

### DETAILED DESCRIPTION

The present disclosure is directed to surface-modified microparticles comprising a microparticle core and at least one monolayer associated with the microparticle core. The microparticle core can includes an active agent. In one aspect, the active agent is a macromolecule. Macromolecules which can also be active agents such as, for example, carbohydrates, peptides, proteins, vectors, nucleic acids, complexes thereof, and conjugates routinely have modifiable functional groups. The monolayer can be a saturated monolayer. More than one monolayer can be present. Typically, each such monolayer comprises at least one charged compound, and at least one monolayer comprises an amphiphilic polymer or a nonionic polymer grafted to an ionic polymer. Most frequently, even when more than one monolayer is included, only a single monolayer comprising an amphiphilic polymer or nonionic polymer grafted to an ionic polymer is present, and said monolayer comprising the grafted polymer generally is disposed such that it comprises the outermost layer of the microparticle. The degree of grafting of the amphiphilic polymer or nonionic polymer to the ionic polymer typically can be from about 1% to about 30%, wherein the degree of grafting is defined as moles of amphiphilic polymer or nonionic polymer divided by moles of modifiable functional groups of the ionic polymer.

Unless otherwise defined herein, scientific and technical terminologies employed in the present disclosure have the meanings that are commonly understood and used by one of ordinary skill in the art. Unless otherwise required by context, singular terms include plural forms of the same and plural terms include the singular. Specifically, as used herein and in the claims, the singular forms “a” and “an” include the plural reference unless the context clearly indicates otherwise. Thus, for example, the reference to a particular microparticle is a reference to one such microparticle or a plurality of such microparticles, including equivalents thereof known to one skilled in the art. Also, as used herein and in the claims, the terms “at least one” and “one or more” have the same meaning and include one, two, three or more. The following terms, unless otherwise indicated, are understood to have the following meanings when used in the context of the present disclosure.

“Microparticle” refers to a particulate that is solid (including substantially solid or semi-solid), having an average geometric particle size (sometimes referred to as diameter) of less than 1 mm, preferably 200 microns or less, more preferably 100 microns or less, most preferably 10 microns or less. In one example, the particle size may be 0.01 microns or greater, preferably 0.1 microns or greater, more preferably 0.5 microns or greater, and most preferably from 0.5 microns to 5 microns. Average geometric particle size may be measured by dynamic light scattering methods (such as photocorrelation spectroscopy, laser diffraction, low-angle laser light scattering (LALLS), and medium-angle laser light scattering (MALLS)), light obscuration methods (such as Coulter analysis method), or other methods (such as rheology, and light or electron microscopy). Particles for pulmonary delivery will typically have an aerodynamic particle size determined by time of flight measurements or Andersen Cascade Impactor measurements. Microparticles may have a spherical shape (sometimes referred to as microspheres) and/or may be encapsulated (sometimes referred to as microcapsules). Certain microparticles may have one or more internal voids and/or cavities. Other microparticles may be free of such voids or cavities. Microparticles may be porous or non-porous. Microparticles may be formed from, in part or in whole, one or more non-limiting materials, such as active agents, carriers, polymers, stabilizing agents, and/or complexing agents.

“Microparticle core” refers to a microparticle fabricated using one or more non-limiting methods, such as those known to one skilled in the art, without surface modification as described herein. The microparticle cores have or are capable of having on their outer surface a net surface electric charge that is positive, negative, or neutral. The microparticle core typically comprises one or more active agents and, optionally, one or more carriers, which, independently, may be compartmentalized in a portion of the microparticle core or preferably be distributed substantially homogeneously throughout the microparticle core. The net surface charge, preferably being non-zero, may be contributed primarily or at least, substantially, by the active agent(s) and/or the optional carrier(s).

“Grafted” or “grafting” refers to the presence or formation of one or more covalent bonds between two or more molecules.

“Degree of grafting” as used herein refers to the number of moles of amphiphilic polymer or non-ionic polymer divided by the number of moles of modifiable functional groups of the ionic polymer, up to a maximum of 100%. The moles of amphiphilic polymer or nonionic polymer equals the total moles of amphiphilic polymer or nonionic polymer provided in the grafting reaction with the ionic polymer. The moles of modifiable functional groups of the ionic polymer equals the total moles of ionic polymer provided in the grafting reaction multiplied by the number of modifiable functional groups present on the ionic polymer. Typically, the ionic polymer will comprise one modifiable functional group per monomer, and therefore the moles of modifiable functional groups will equal the moles of monomer. For example, if the ionic polymer typically has a chain length of 8 monomers, 8 moles of modifiable functional groups are present on the polymer. Thus, as used herein, the degree of grafting describes a grafted ionic polymer wherein the amphiphilic polymer or nonionic polymer has been assumed to react completely with the ionic polymer.

“Active agent” refers to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. The active agent may be capable of preventing, alleviating, treating, and/or curing abnormal and/or pathological conditions of a living body, such as by destroying a parasitic organism, or by limiting the effect of a disease or abnormality by materially altering the physiology of the host or parasite. The active agent may be capable of maintaining, increasing, decreasing, limiting, or destroying a physiologic body function. The active agent may be capable of diagnosing a physiological condition or state by an in vitro and/or in vivo test. The active agent may be capable of controlling or protecting an environment or living body by attracting, disabling, inhibiting, killing, modifying, repelling and/or retarding an animal or microorganism. The active agent may be capable of otherwise treating (such as deodorizing, protecting, adorning, grooming) a body. Depending on the effect and/or its application, the active agent may further be referred to as a bioactive agent, a pharmaceutical agent (such as a prophylactic agent, or a therapeutic agent), a diagnostic agent, a nutritional supplement, and/or a cosmetic agent, and includes, without limitation, prodrugs, affinity molecules, synthetic organic molecules, polymers, molecules with a molecular weight of 2 kD or less (such as 1.5 kD or less, or 1 kD or less), macromolecules (such as those having a molecular weight of 2 kD or greater, preferably 5 kD or greater), proteinaceous compounds, peptides, vitamins, steroids, steroid analogs, lipids, nucleic acids, carbohydrates, precursors thereof and derivatives thereof. Active agents may be ionic, non-ionic, neutral, positively charged, negatively charged, or zwitterionic, and may be used singly or in combination of two or more thereof. Active agents may be water insoluble, or water soluble. Active agents may have an isoelectric point of 7.0 or greater, or less than 7.0.

“Macromolecule” refers to a material capable of providing a three-dimensional (e.g., tertiary and/or quaternary) structure, and includes carriers and certain active agents of the present disclosure. Macromolecules typically have a molecular weight of 2 kD or greater, preferably 5 kD or greater. Non-limiting macromolecules used to form the microparticles include, inter alia, polymers, copolymers, proteins (e.g., enzymes, recombinant proteins, albumins such as human serum albumin, monoclonal antibodies, polyclonal antibodies), peptides, lipids, carbohydrates (e.g., monosaccharides, disaccharides, polysaccharides), nucleic acids, vectors (e.g., virus, viral particles), and complexes and conjugates thereof (e.g., covalent and/or non-covalent associations between two macromolecules such as carbohydrate-protein complexes or conjugates, or between an active agent and a macromolecule such as hapten-protein complexes or conjugates). Macromolecules may be neutral, positively charged, negatively charged, or zwitterionic, and may be used singly or in combination of two or more thereof.

“Proteinaceous compounds” refer to natural, synthetic, semi-synthetic, or recombinant compounds of or related structurally and/or functionally to proteins, such as those containing or consisting essentially of α-amino acids covalently associated through peptide linkages. Non-limiting proteinaceous compounds include globular proteins (e.g., albumins, globulins, histones), fibrous proteins (e.g., collagens, elastins, keratins), compound proteins (including those containing one or more non-peptide component, e.g., glycoproteins, nucleoproteins, mucoproteins, lipoproteins, metalloproteins), therapeutic proteins, fusion proteins, receptors, antigens (such as synthetic or recombinant antigens), viral surface proteins, hormones and hormone analogs, antibodies (such as monoclonal or polyclonal antibodies), enzymes, Fab fragments, cyclic peptides, linear peptides, and the like. Non-limiting therapeutic proteins include bone morphogenic proteins, drug resistance proteins, toxoids, erythropoietins, proteins of the blood clotting cascade (e.g., Factor VII, Factor VIII, Factor IX), subtilisin, ovalbumin, alpha-1-antitrypsin (AAT), DNase, superoxide dismutase (SOD), lysozyme, ribonuclease, hyaluronidase, collagenase, human growth hormone (hGH), erythropoietin, insulin and insulin-like growth factors or their analogs, interferons, glatiramer, granulocyte-macrophage colony-stimulating factor, granulocyte colony-stimulating factor, desmopressin, leutinizing hormone release hormone (LHRH) agonists (e.g., leuprolide, goserelin, buserelin, gonadorelin, histrelin, nafarelin, deslorelin, fertirelin, triptorelin), LHER antagonists, vasopressin, cyclosporine, calcitonin, parathyroid hormone, parathyroid hormone peptides, insulin, glucogen-like peptides, and analogs thereof. Proteinaceous compounds may be neutral, positively charged, negatively charged, or zwitterionic, and may be used singly or in combination of two or more thereof.

“Peptides” refer to natural, synthetic, or semi-synthetic compounds formed at least in part from two or more of the same or different amino acids and/or imino acids. Non-limiting examples of peptides include oligopeptides (such as those having less than 50 amino/imino acid monomer units, including dipeptides and tripeptides and the like), polypeptides, proteinaceous compounds as defined herein, as well as precursors and derivatives thereof (e.g., glycosylated, hyperglycosylated, PEGylated, FITC-labeled, and salts thereof). Peptides may be neutral, positively charged, negatively charged, or zwitterionic, and may be used singly, or in combination of two or more thereof.

“Lipids” refer to natural, synthetic, or semi-synthetic compounds that are generally amphiphilic. The lipids typically comprise a hydrophilic component and a hydrophobic component. Non-limiting examples include fatty acids, neutral fats, phosphatides, oils, glycolipids, surfactants, aliphatic alcohols, waxes, terpenes and steroids. Lipids may be ionic, non-ionic, neutral, positively charged, negatively charged, or zwitterionic, and may be used singly or in combination of two or more thereof.

“Nucleic acids” refer to natural, synthetic, semi-synthetic, or recombinant compounds formed at least in part from two or more of the same or different nucleotides, and may be single-stranded or double-stranded. Non-limiting examples of nucleic acids include oligonucleotides (such as those having 20 or less base pairs, e.g., sense, anti-sense, or missense), aptamers, polynucleotides (e g., sense, anti-sense, or missense), DNA (e.g., sense, anti-sense, or missense), RNA (e.g., sense, anti-sense, or missense), siRNA, nucleotide acid constructs, single-stranded or double-stranded segments thereof, as well as precursors and derivatives thereof (e.g., glycosylated, hyperglycosylated, PEGylated, FITC-labeled, nucleosides, and salts thereof). Nucleic acids may be neutral, positively charged, negatively charged, or zwitterionic, and may be used singly or in combination of two or more thereof.

“Carbohydrates” refer to natural, synthetic, or semi-synthetic compounds formed at least in part from monomeric sugar units. Non-limiting carbohydrates include polysaccharides, sugars, starches, and celluloses, such as carboxymethylcellulose, dextrans, hetastarch, cyclodextrins, alginates, chitosans, chondroitins, heparins, as well as precursors and derivatives thereof (e.g., glycosylated, hyperglycosylated, PEGylated, FITC-labeled, and salts thereof). Carbohydrates may be ionic or non-ionic, may be neutral, positively charged, negatively charged, or zwitterionic, and may be used singly or in combination of two or more thereof.

“Carrier” refers to a compound, typically a macromolecule, having a primary function to provide a three-dimensional structure (including tertiary and/or quaternary structure) The carrier may be unassociated or associated with the active agent (such as conjugates or complexes thereof) in forming microparticles as described above. The carrier may further provide other functions, such as being an active agent, modify release profile of the active agent from the microparticle, and/or impart one or more particular properties to the microparticle (such as contribute at least in part to the net surface charge). In one example, the carrier is a protein (such as albumins, preferably human serum albumin) having a molecular weight of 1500 Daltons or greater, for example, 2000 Daltons or greater.

“Polymer” or “polymeric” refers to a natural, synthetic, or semi-synthetic molecule having in a main chain or ring structure two or more repeating monomer units. Polymers broadly include dimers, trimers, tetramers, oligomers, higher molecular weight polymer, adducts, homopolymers, random copolymers, pseudo-copolymers, statistical copolymers, alternating copolymers, periodic copolymer, bipolymers, terpolymers, quaterpolymers, other forms of copolymers, substituted derivatives thereof, and mixtures thereof, and narrowly refer to molecules having 10 or more repeating monomer units. Polymers may be linear, branched, block, graft, monodisperse, polydisperse, regular, irregular, tactic, isotactic, syndiotactic, stereoregular, atactic, stereoblock, single-strand, double-strand, star, comb, dendritic, and/or ionomeric. Polymers may be ionic, non-ionic, neutral, positively charged, negatively charged, or zwitterionic, and may be used singly or in combination of two or more thereof.

“Stabilizing,” used especially in conjunction with an agent (e.g., compound), a process, or a condition, refers to the capability of such agent, process or condition to, at least in part, form the microparticles (or a composition or formulation or kit containing such microparticles), facilitate the formation thereof, and/or enhance the stability thereof (e.g., the maintenance of a relatively balanced condition, like increased resistance against destruction, decomposition, degradation, and the like). Non-limiting stabilizing processes or conditions include thermal input/output (e.g., heating, cooling), electromagnetic irradiation (e.g., gamma rays, X rays, UV, visible light, actinic, infrared, microwaves, radio waves), high-energy particle irradiation (e.g., electron beams, nuclear), and ultrasound irradiation. Non-limiting stabilizing agents include lipids, proteins, polymers, carbohydrates, surfactants, salts (e.g., organic, inorganic, comprising cations that are monovalent or multivalent, comprising cations that are organic, metallic, or organometallic, comprising anions that are monovalent or multivalent, and comprising anions that are organic, inorganic, or organometallic), as well as certain of the carriers, the active agents, the crosslinkers, the co-agents, and the complexing agents disclosed herein. The stabilizing agents may be ionic, non-ionic, neutral, positively charged, negatively charged, or zwitterionic, and may be used singly or in combination of two or more thereof.

“Complexing agent” refers to a material capable of forming one or more non-covalent associations with the active agent. Through such associations, the complexing agent is capable of facilitating the loading of one or more active agents into the microparticle, retaining the active agent(s) within the microparticle, and/or otherwise modifying the release of the active agent(s) from the microparticle. Complexing agents may be ionic, non-ionic, neutral, positively charged, negatively charged, or zwitterionic, and may be used singly or in combination of two or more thereof.

“Charged” and “electrically charged” refer interchangeably to the capability of providing one, two, three, or more formal units of electrical charges of the same or opposite sign and/or the presence of such charges (i.e., “charged” refers to chargeable and/or charged).

“Charged compound” and “electrically charged compound” refer interchangeably to a single compound that is charged as described above, or a combination of two or more different compounds in unassociated and/or associated forms (e.g., conjugates, aggregates, and/or complexes thereof), each of which independently has and/or is capable of having a net charge of the same sign.

“Net charge” and “net electric charge” are used interchangeably and refer to the sum of all formal units of electric charge a charged compound is capable of having or has, such as in a flowable medium under certain conditions (preferably in a solution of certain pH). The net charge may be positive, negative, or zero (such as in zwitterionic compounds), and is condition-dependent (e.g., solvent-dependent, or pH-dependent).

“Net surface charge” and “net surface electric charge” are used interchangeably and refer to an overall cumulative electric charge on an outermost surface of a three-dimensional structure (e.g., a microparticle, or a monolayer). The net surface charge may be positive, negative, or zero, and is condition-dependent (e.g., solvent-dependent, or pH-dependent).

“Spherical” refers to a geometric shape that is at least “substantially spherical.” “Substantially spherical” means that the ratio of the longest length (i.e., one between two points on the perimeter and passes the geometric center of the shape) to the shortest length on any cross-section that passes through the geometric center is about 1.5 or less, preferably about 1.33 or less, more preferably 1.25 or less. Spherical does not require a line of symmetry. Further, the microparticles may have surface texturing (such as continuous or discrete lines, islands, lattices, indentations, channel openings, or protuberances that are small in scale when compared to the overall size of the microparticles) and still be spherical. Surface contact is minimized between microparticles that are spherical, which minimizes the undesirable agglomeration of the microparticles. In comparison, microparticles that are crystals or flakes typically display significant agglomeration through ionic and/or non-ionic interactions at relatively large flat surfaces.

“Monodisperse size distribution” refers to a preferred microparticle size distribution in which the ratio of the volume diameter of the 90th percentile (i.e., the average particle size of the largest 10% of the microparticles) to the volume diameter of the 10th percentile (i.e., the average particle size of the smallest 10% of the microparticles) is about 5 or less, preferably about 3 or less, more preferably about 2 or less, most preferably about 1.5 to 1. Consequently, “polydisperse size distribution” refers to one where the diameter ratio described above is greater than 5, preferably greater than 8, more preferably greater than 10. In microparticles having a polydisperse size distribution, smaller microparticles may fill in the gaps between larger microparticles, thus possibly displaying large contact surfaces and significant agglomeration there between. A Geometric Standard Deviation (GSD) of 2.5 or less, preferably 1.8 or less, may also be used to indicate a monodisperse size distribution. Calculation of GSD is known and understood to one skilled in the art.

“Amorphous” refers to materials and constructions that are “substantially amorphous,” such as microparticles having multiple non-crystalline domains (or lacking crystallinity altogether) or otherwise non-crystalline. Substantially amorphous microparticles of the present disclosure are generally random solid particulates in which crystalline lattices constitute less than 50% by volume and/or weight of the microparticles, or are absent, and include semi-crystalline microparticles and non-crystalline microparticles as understood by one skilled in the art.

“Suspension” or “dispersion” refers to a mixture, preferably finely divided, of two or more phases (e g., solid, liquid, gas), such as solid in liquid, liquid in liquid, gas in liquid, solid in solid, solid in gas, liquid in gas, and the like. The suspension or dispersion may preferably remain stable for extended periods of time (e.g., minutes, hours, days, weeks, months, years).

“Resuspending” refers to changing microparticles from a non-flowable (e.g., solid) state to a flowable (e.g., liquid) state by adding a flowable medium (e.g., a liquid), while retaining most or all of the characteristics of the microparticles. The liquid may be, for example, aqueous, aqueous miscible, or organic.

“Monolayer” refers to a single layer or coating formed over a three-dimensional substrate, from a composition of one or more compounds (such as a charged compound as described above). The monolayer may be a continuous and nonporous monolayer, a continuous and porous monolayer (such as a lattice network), a non-continuous monolayer of a plurality of discrete elements (e.g., islands, strips, clusters, etc.), or a combination thereof. While not intending to be bound by theory, typically the monolayer will be decomposable or degradable, such as biodegradable, enzymatically or hydrolytically degradable and the like, to allow for non-diffusional release of an active agent (from the microparticle) over which the monolayer is deposited. The monolayer may have a thickness of 100 nm or less, preferably 50 nm or less, more preferably 20 nm or less, most preferably 10 nm or less. In one example, the monolayer is formed through self-assembly of a charged compound.

“Saturated monolayer” refers to a monolayer as defined above that is incapable of further incorporating, cumulatively, an excess amount of the composition forming the monolayer when subjected to the same set of conditions under which the monolayer is formed.

“Therapeutic” refers to any pharmaceutic, drug, prophylactic agent, contrast agent, or dye useful in the treatment (including prevention, diagnosis, alleviation, suppression, remission, or cure) of a malady, affliction, disease or injury in a subject. Therapeutically useful peptides and nucleic acids may be included within the meaning of the term “pharmaceutic” or “drug.”

“Affinity molecule” refers to any material or substance capable of promoting binding and/or targeting of regions in vivo and/or tissues/receptors in vitro. Affinity molecules, including receptors and targeting ligands, may be natural, synthetic, or semi-synthetic, may be ionic or non-ionic, may be neutral, positively charged, negatively charged, or zwitterionic, and may be used singly or in combination of two or more thereof. Non-limiting affinity molecules include proteinaceous compounds (e.g., antibodies, antibody fragments, hormones, hormone analogs, glycoproteins, and lectins), peptides, polypeptides, amino acids, sugars, saccharides (e.g. monosaccharides, polysaccharides, carbohydrates), vitamins, steroids, steroid analogs, cofactors, active agents, nucleic acids, viruses, bacteria, toxins, antigens, other ligands, and precursors and derivatives thereof.

“Receptor” refers to a molecular structure within a cell or on its surface, generally characterized by its selective binding of a specific substance, e.g., ligand. Non-limiting receptors include cell-surface receptors for peptide hormones, neurotransmitters, antigens, complement fragments, and immunoglobulins, and cytoplasmic receptors for steroid hormones.

“Precursor” refers to any material or substance capable of being converted to a desired material or substance, preferably through a chemical and/or biochemical reaction or pathway, such as anchoring a precursor to a material. Non-limiting precursor moieties include maleimide groups, disulfide groups (e.g., ortho-pyridyl disulfide), vinylsulfone groups, azide groups, and α-iodoacetyl groups.

“Derivative” refers to any material or substance formed from a parent material or substance, preferably through a chemical and/or biochemical reaction or pathway considered routine by one of ordinary skill in the art. Non-limiting examples of derivatives include glycosylated, hyperglycosylated, PEGylated, FITC-labelled, protected with protecting groups (e.g., benzyl for alcohol or thiol, t-butoxycarbonyl for amine), as well as salts, esters, amides, conjugates, complexes, manufacturing related compounds, and metabolites thereof. Salts may be organic, inorganic, comprising cations that are monovalent or multivalent, comprising cations that are organic, metallic, or organometallic, comprising anions that are monovalent or multivalent, and comprising anions that are organic, inorganic, or organometallic. Preferred salts are pharmaceutically acceptable, and include, without limitation, mineral or organic acid salts of basic residues (e.g., amines), alkali or organic salts of acidic residues (e.g., carboxylic acids), and the like, such as conventional nontoxic salts or the quaternary ammonium salts of the parent compound formed from non-toxic inorganic acids (e.g., hydrochloric, hydrobromic, sulfuric, sulfamic, phosphoric, nitric) and organic acids (e.g., acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic).

“Analog” refers to a compound having a chemically modified form of a principle compound or class thereof, which maintains the pharmaceutical and/or pharmacological activities characteristic of the principle compound or class.

“Prodrug” refers to any covalently bonded carriers that release an active agent in vivo when administered to a subject. Prodrugs are known to enhance numerous desirable qualities (e.g., solubility, bioavailability, manufacturing) of the active agents. Prodrugs may be prepared by modifying functional groups (e.g., hydroxy, amino, carboxyl, and/or sulfhydryl groups) present in the active agent in such a way that the modifications are reversed (e g., modifier group cleaved), either in routine manipulation or in vivo, to afford the original active agent. The transformation in vivo may be, for example, the result of some metabolic process, such as chemical or enzymatic hydrolysis of a carboxylic, phosphoric or sulfate ester, or reduction or oxidation of a susceptible functionality.

“Metabolite” refers to a form of a compound obtained in a subject body by action of the body on the administered form of the compounds. For example, a demethylated metabolite may be obtained in the body after administration of a methylated compound bearing a methyl group. Metabolites may themselves have biological, preferably therapeutic, activities.

“Diagnostic agent” refers to any material or substance useful in connection with methods for perceptually observing (e.g., imaging) a normal or abnormal biological condition or state, or detecting the presence or absence of a pathogen or a pathological condition. Non-limiting diagnostic agents include contrast agents and dyes for use in connection with radiography imaging (e.g., X-ray imaging), ultrasound imaging, magnetic resonance imaging, computed tomography, positron emission tomography imaging, and the like. Diagnostic agents further include any other agents useful in facilitating diagnosis in vivo and/or in vitro, whether or not imaging methodology is employed.

“Cross-link,” “cross-linked” and “cross-linking” generally refer to the linking of two or more materials and/or substances, including any of those disclosed herein, through one or more covalent and/or non-covalent (e.g., ionic) associations. Cross-linking may be effected naturally (e.g., disulfide bonds of cysteine residues) or through synthetic or semi-synthetic routes, for example, optionally in the presence of one or more cross-linkers (i.e., a molecule X by itself capable of reacting with two or more materials/substances Y and Z to form a cross-link product Y-X-Z, where the associations of Y-X and X-Z are independently covalent and/or non-covalent), initiators (i.e., a molecule by itself capable of providing reactive species like free radicals for the cross-link reaction, e g., thermally decomposable initiators like organic peroxides, azo initiators, and carbon-carbon initiators, actinically decomposable initiators like photoinitiators of various wavelengths), activators (i.e., a molecule capable of reacting with a first material/substance Y to form an activated intermediate [A-Y], which in turn reacts with a second material/substance Z to form a cross-link product Y-Z, while A is chemically altered or consumed during the process), catalysts (i.e., a molecule capable of modifying the kinetics of the cross-link reaction without being chemically modified during the process), co-agents (i.e., a molecule that, when co-present with one or more of the initiators, activators, and/or catalysts, is capable of modifying the kinetics of the cross-link reaction and/or being incorporated into the cross-link product of the two or more materials/substances, but otherwise is non-reactive to the materials/substances), and/or energy sources (e.g., heating; cooling; high-energy radiations such as electromagnetic, electron beams, and nuclear; acoustic radiations such as ultrasonic).

“In association with” and “associated with” refer in general to the one or more interactions between different materials (typically those that are part of the microparticles), such as between one or more of such materials and/or one or more structures (or portions thereof) of the microparticles. The materials of the microparticles include, without limitation, ions such as monovalent and multivalent ions disclosed herein, as well as compounds such as active agents, stabilizing agents, cross-linking agents, charged or uncharged compounds, the various polymers disclosed herein, and combinations of two or more thereof. The structures of the microparticles and portions thereof include, without limitation, core, microparticle core, monolayer, intermediate microparticle, surface-modified microparticle, portions of such structures (such as outer surfaces, inner surfaces), domains between such structures and portions thereof, and combinations of two or more thereof. The various associations may be reversible or irreversible, and may be present singly or in combination of two or more thereof. Non-limiting associations include covalent and/or non-covalent associations (e.g., covalent bonding, ionic interactions, electrostatic interactions, dipole-dipole interactions, hydrogen bonding, van der Waal's forces, cross-linking, and/or any other interactions), encapsulation in a layer, compartmentalization between two layers, interspersion, conjugation, and/or complexation between different materials. As used herein, all monolayers of a surface-modified microparticle are associated with the microparticle core, including, for example, when multiple monolayers are present, an innermost monolayer, an outermost monolayer, and any monolayers between the innermost and outermost layers.

“Covalent association” refers to an intermolecular interaction (e.g., a bond) between two or more individual molecules that involves the sharing of electrons in the bonding orbitals of two atoms.

“Non-covalent association” refers to an intermolecular interaction between two or more individual molecules without involving a covalent bond. Intermolecular interaction depends on, for example, polarity, electric charge, and/or other characteristics of the individual molecules, and includes, without limitation, electrostatic (e.g., ionic) interactions, dipole-dipole interactions, van der Waal's forces, and combinations of two or more thereof.

“Electrostatic interaction” refers to an intermolecular interaction between two or more positively or negatively charged moieties/groups, which may be attractive when two are oppositely charged (i.e., one positive, another negative), repulsive when two charges are of the same sign (i.e., two positive or two negative), or a combination thereof.

“Dipole-dipole interaction” refers an intermolecular attraction between two or more polar molecules, such as a first molecule having an uncharged, partial positive end δ⁺ (e.g., electropositive head group such as the choline head group of phosphatidylcholine) and a second molecule having an uncharged, partial negative end δ⁻ (e.g., an electronegative atom such as the heteroatom O, N, or S in a polysaccharide). Dipole-dipole interaction also refers to intermolecular hydrogen bonding in which a hydrogen atom serves as a bridge between electronegative atoms on separate molecules and in which a hydrogen atom is held to a first molecule by a covalent bond and to a second molecule by electrostatic forces.

“Hydrogen bond” refers to an attractive force or bridge between a hydrogen atom covalently bonded to a first electronegative atom (e.g., O, N, S) and a second electronegative atom, wherein the first and second electronegative atoms may be in two different molecules (intermolecular hydrogen bonding) or in a single molecule (intramolecular hydrogen bonding).

“Van der Waal's forces” refers to the attractive forces between non-polar molecules that are accounted for by quantum mechanics. Van der Waal's forces are generally associated with momentary dipole moments induced by neighboring molecules undergoing changes in electron distribution.

“Hydrophilic interaction” refers to an attraction toward water molecules, wherein a material/compound or a portion thereof may bind with, absorb, and/or dissolve in water. This may result in swelling and/or the formation of reversible hydrogels.

“Hydrophobic interaction” refers to a repulsion against water molecules, wherein a material/compound or a portion thereof does not bind with, absorb, or dissolve in water.

“Biocompatible” refers to materials/substances that are generally not injurious to biological functions and do not result in unacceptable toxicity (e.g., allergenic responses or disease states).

“Subject” or “patient” refers to animals, including vertebrates like mammals, preferably humans.

“Region of a subject” refers to a localized internal or external area or portion of the subject (e.g., an organ), or a collection of areas or portions throughout the entire subject (e.g., lymphocytes). Non-limiting examples of such regions include pulmonary region (e.g., lung, alveoli, gastrointestinal region (e.g., regions defined by esophagus, stomach, small and large intestines, and rectum), cardiovascular region (e.g., myocardial tissue), renal region (e.g., the region defined by the kidney, the abdominal aorta, and vasculature leading directly to and from the kidney), vasculature (i.e., blood vessels, e.g., arteries, veins, capillaries, and the like), circulatory system, healthy or diseased tissues, benign or malignant (e.g., tumorous or cancerous) tissues, lymphocytes, receptors, organs and the like, as well as regions to be imaged with diagnostic imaging, regions to be administered and/or treated with an active agent, regions to be targeted for the delivery of an active agent, and regions of elevated temperature.

“Tissue” refers generally to an individual cell or a plurality or aggregate of cells specialized and capable of performing one or more particular functions. Non-limiting tissue examples include membranous tissues, (e.g., endothelium, epithelium), blood, laminae, connective tissue (e.g., interstitial tissue), organs (e.g., myocardial tissue, myocardial cells, cardiomyocites), and abnormal cell(s) (e.g., tumors).

“Ambient temperature” refers to a temperature of around room temperature, typically in a range of about 20° C. to about 40° C., for example, about 20° C. to about 25° C.

“Controlled release” refers to an altered in vivo and/or in vitro release (e.g., dissolution) profile of an active agent, as compared to the release profile of the active agent in its native form or, for example, relative to an unmodified microparticle. The active agent is preferably associated with a microparticle or a composition or formulation containing such a microparticle, as disclosed herein, such that one or more aspects of its release kinetics (e.g., initial burst, quantity and/or rate over a specified time period or phase, cumulative quantity over a specific time period, length of time for total release, pattern and/or profile, etc.) are increased, decreased, shortened, prolonged, and/or otherwise modified as desired. Non-limiting examples of controlled release include immediate/instant release (i.e., initial burst or rapid release), extended release, sustained release, prolonged release, delayed release, modified release, and/or targeted release, occurring individually, or in combination of two or more thereof.

“Extended release” refers to the release of an active agent, preferably in association with a microparticle or a composition or formulation containing such a microparticle, as disclosed herein, over a time period longer than the free aqueous diffusion period of the active agent in its native form or, for example, relative to an unmodified microparticle. The extended release period may be hours (e.g., at least about 1, 2, 5, or 10 hours), days (e.g., at least about 1, 2, 3, 4, 5, 6, 7, 8, 10, 15, 20, 30, 40, 45, 60, or 90 days), weeks (at least about 1, 2, 3, 4, 5, 6, 10, 15, 20, 30, 40, or 50 weeks), months (at least about 1, 2, 3, 4, 6, 9, or 12 months), about 1 or more years, or a range between any two of the time periods. The pattern of an extended release may be continuous, periodic, sporadic, or a combination thereof.

“Sustained release” refers to an extended release of an active agent such that a functionally significant level of the active agent (i.e., a level capable of bringing about the desired function of the active agent) is present at any time point of the extended release period, preferably with a continuous and/or uniform release pattern. Non-limiting examples of sustained release profiles include those, when displayed in a plot of release time (x-axis) versus cumulative release (y-axis), showing at least one upward segment that is linear, step-wise, zig-zagging, curved, and/or wavy, over a time period of 1 hour or longer.

Examples provided herein, including those following “such as” and “e.g.,” are considered as illustrative only of various aspects of the present disclosure and embodiments thereof, without being specifically limited thereto. Any suitable equivalents, alternatives, and modifications thereof (including materials, substances, constructions, compositions, formulations, means, methods, conditions, etc.) known and/or available to one skilled in the art may be used or carried out in place of or in combination with those disclosed herein, and are considered to fall within the scope of the present disclosure.

### The Microparticle Core

Methods of making the microparticle cores are not particularly limited, and include those disclosed in U.S. Pat. Nos. 5,525,519, 5,554,730, 5,578,709, 5,599,719, 5,981,719, 6,090,925, 6,268,053, and 6,458, 387, U.S. Publication Nos. 20030059474, 20030064033, 20040043077, 20050048127, 20050142201, 20050142205, 20050142206, 20050147687, 20050170005, 20050233945, 20060018971, 20060024240, 20060024379, 20060260777, 20070092452, 20070207210, and 20070281031, the disclosures of which are herein incorporated by reference in their entirety. In one example, a single flowable continuous phase system (such as liquid, gas, or plasma, preferably a solution or suspension) is formulated to contain one or more active agents, a medium, and one or more phase-separation enhancing agents (PSEAs). The medium is preferably a liquid solvent (e.g., hydrophilic or hydrophobic organic solvents, water, buffers, aqueous-miscible organic solvents, and combinations of two or more thereof), more preferably an aqueous or aqueous-miscible solvent. Suitable organic solvents include, without limitation, methylene chloride, chloroform, acetonitrile, ethyl acetate, methanol, ethanol, pentane, the likes thereof, and combinations of two or more thereof (such as a 1:1 mixture of methylene chloride and acetone). The active agent and the PSEA may independently be dissolved, suspended, or otherwise homogeneously distributed within the medium. When subjecting the flowable system to certain conditions (such as a temperature below the phase transition temperature of the active agent in the medium), the active agent undergoes a liquid-solid phase separation and forms a discontinuous, preferably solid, phase (such as a plurality of microparticle cores suspended in the medium), while the PSEA remains in the continuous phase (such as being dissolved in the medium).

The medium can be organic, for example, containing an organic solvent or a mixture of two or more inter-miscible organic solvents, which may independently be aqueous-miscible or aqueous-immiscible. The solution can also be an aqueous-based solution containing an aqueous medium, an aqueous-miscible organic solvent, a mixture of aqueous-miscible organic solvents, or combinations thereof. The aqueous medium can be water, a buffer (e.g., normal saline, buffered solutions, buffered saline), and the like. Suitable aqueous-miscible organic solvents may be monomers or polymers, and include, but are not limited to, N-methyl-2-pyrrolidinone (N-methyl-2-pyrrolidone), 2-pyrrolidinone (2-pyrrolidone), 1,3-dimethyl-2-imidazolidinone (DMI), dimethylsulfoxide, dimethylacetamide, acetic acid, lactic acid, acetone, methyl ethyl ketone, acetonitrile, methanol, ethanol, n-propanol, isopropanol, 3-pentanol, benzyl alcohol, glycerol, tetrahydrofuran (THF), polyethylene glycol (PEG, e.g., PEG-4, PEG-8, PEG-9, PEG-12, PEG-14, PEG-16, PEG-120, PEG-75, PEG-150), PEG esters (e.g., PEG-4 dilaurate, PEG-20 dilaurate, PEG-6 isostearate, PEG-8 palmitostearate, PEG-150 palmitostearate), PEG sorbitans (such as PEG-20 sorbitan isostearate), PEG ethers (such as monoalkyl and dialkyl ethers, e.g., PEG-3 dimethyl ether, PEG-4 dimethyl ether, and glycofurol), polypropylene glycol (PPG), PPG esters (such as polypropylene glycol alginate (PGA), PPG dicaprylate, PPG dicaprate, PPG laurate), alkoxylated linear alkyl diols (such as PPG-10 butanediol), alkoxylated alkyl glucose ethers (e.g., PPG-10 methyl glucose ether, PPG-20 methyl glucose ether), PPG alkyl ethers (such as PPG-15 stearyl ether), alkanes (e.g., propane, butane, pentane, hexane, heptane, octane, nonane, decane), and combinations of two or more thereof.

In a preferred example, a solution of the PSEA in a first solvent is provided, in which the PSEA is soluble in or miscible with the first solvent. The active agent is mixed in, directly or as a second solution in a second solvent, with the first solution. The first and second solvent may be the same or at least miscible with each other. Preferably, the active agent is added at a temperature equal to or lower than ambient temperature, particularly when the active agent is a heat labile molecule such as certain proteinaceous compounds. However, the system may be heated to increase solubility of the active agent in the system, as long as the activity of the active agent is not adversely affected.

When the mixture is brought to phase separation conditions, the PSEA, while remaining in the liquid continuous phase, enhances and/or induces a liquid-solid phase separation of the active agent from the solution (such as by reducing solubility of the active agent), thereby forming the microparticle cores (the solid discontinuous phase), which may preferably be microspheres. Suitable PSEA compounds include, but are not limited to, natural and synthetic polymers, linear polymers, branched polymers, cyclo-polymers, copolymers (random, block, grafted, such as poloxamers, particularly PLURONIC® F127 and F68), terpolymers, amphiphilic polymers, carbohydrate-based polymers, polyaliphatic alcohols, poly(vinyl)polymers, polyacrylic acids, polyorganic acids, polyamino acids, polyethers, polyesters, polyimides, polyaldehydes, polyvinylpyrrolidone (PVP), and surfactants. Suitable or exemplary PSEAs include, without limitation, polymers acceptable as pharmaceutical additives, such as PEGs (e.g., PEG 200, PEG 300, PEG 400, PEG 600, PEG 800, PEG 1000, PEG 3350, PEG 8000, PEG 10000, PEG 20000, etc), poloxamers, PVP, hydroxyethylstarch, amphiphilic polymers, as well as non-polymers (such as mixtures of propylene glycol and ethanol).

Conditions capable of enhancing, inducing, promoting, controlling, suppressing, retarding, or otherwise affecting the liquid-solid phase separation include, without limitation, changes in temperature, pressure, pH, ionic strength and/or osmolality of the solutions, concentrations of the active agent and/or the PSEA, the likes thereof, as well as rates of such changes, and combinations of two or more thereof. Such conditions may desirably be applied before and up to the phase separation, or even during the phase separation. In one example, the system is exposed to a temperature below the phase transition temperature of the active agent therein, alone or in combination with adjustments to the concentrations of the active agent and/or the PSEA, as described in U.S. Patent Application Publication 2005/0142206, the entire disclosure of which is incorporated herein by reference. The rate of temperature drop may be held constant or altered in any controlled manner, for example, within a range of 0.2° C./minute to 50° C./minute, preferably 0.2° C./minute to 30° C./minute. Freezing point depressing agents (FPDAs), used individually or in combination of two or more thereof, may be mixed in the system directly or as solutions (such as aqueous solutions) containing such FPDAs, particularly for systems in which the freezing point is higher than the phase transition temperature of the active agent. Suitable FPDAs include, without limitation, propylene glycol, sucrose, ethylene glycol, alcohols (e.g., ethanol, methanol), and aqueous mixtures thereof.

In one example, the microparticle cores may further comprise one or more excipients that negligibly affect the phase separation. The excipient may imbue the microparticle cores and/or the compounds therein (e.g., the active agent, the optional carrier) with additional characteristics such as increased stability, controlled release of the active agent from the microparticle cores, and/or modified permeation of the active agent through biological tissues. Suitable excipients include, but are not limited to, carbohydrates (e.g., trehalose, sucrose, mannitol), multivalent cations (preferably metal cations, e.g., Zn²⁺, Mg²⁺, Ca²⁺, Cu²⁺, Fe²⁺, Fe³⁺), anions (e.g., CO₃²⁻, SO₄²), amino acids (such as glycine), lipids, phospholipids, fatty acids and esters thereof, surfactants, triglycerides, bile acids and conjugates and salts thereof (e.g., cholic acid, deoxycholic acid, glycocholate, taurocholate, sodium cholate), and any polymers disclosed herein.

In one example, the microparticle cores are optionally separated from the solution and washed prior to the surface modification as disclosed herein, or are surface-modified without separation or washing. Separation means include, without limitation, centrifugation, dialysis, sedimentation (creaming), phase separation, chromatography, electrophoresis, precipitation, extraction, affinity binding, filtration, and diafiltration. For active agents with relatively low aqueous solubility, the washing medium may be aqueous, optionally containing one or more solubility reducing agents (SRAs) and/or excipients as disclosed herein. Preferred SRAs are capable of forming insoluble complexes with the active agents and/or carriers in the microparticles, and include, without limitation, compounds such as salts that comprise divalent or multivalent cations (such as those disclosed herein). For active agents with relatively high aqueous solubility (such as proteinaceous compounds), the washing medium may be organic, or aqueous but containing at least one SRA or precipitating agent (such as ammonium sulfate). In one example, the washing medium is the same solution used in the phase separation reaction, such as an aqueous solution including approximately 16% (w/v) PEG and 0.7% (w/v).

It is preferred that the washing medium has a low boiling point for easy removal by, for example, lyophilization, evaporation, or drying. The washing medium may be a supercritical fluid or a fluid near its supercritical point, used alone or in combination with a co-solvent. Supercritical fluids may be solvents for the PSEAs, but not for the microparticle cores. Non-limiting examples of supercritical fluids include liquid CO₂, ethane, and xenon. Non-limiting examples of co-solvents for use with such supercritical fluids include acetonitrile, dichloromethane, ethanol, methanol, water, and 2-propanol.

In one example, the microparticles described herein comprise active agents with varying degrees of solubility in water. Both water insoluble active agents and water soluble active agents are encompassed by the present disclosure.

In one example, the active agent is a pharmaceutical agent. Depending on its effect and/or application, the pharmaceutical agent includes, without limitation, adjuvants, adrenergic agents, adrenergic blocking agents, adrenocorticoids, adrenolytics, adrenomimetics, alkaloids, alkylating agents, allosteric inhibitors, anabolic steroids, analeptics, analgesics, anesthetics, anorexiants, antacids, anthelmintics, anti-allergic agents, antiangiogenesis agents, anti-arrhythmic agents, anti-bacterial agents, antibiotics, antibodies, anticancer agents, anticholinergic agents, anticholinesterases, anticoagulants, anticonvulsants, antidementia agents, antidepressants, antidiabetic agents, antidiarrheals, antidotes, antiepileptics, antifolics, antifungals, antigens, antihelmintics, antihistamines, antihyperlipidemics, antihypertensive agents, anti-infective agents, anti-inflammatory agents, antimalarials, antimetabolites, antimuscarinic agents, antimycobacterial agents, antineoplastic agents, antitumor agents, antiosteoporosis agents, antipathogen agents, antiprotozoal agents, adhesion molecules, antipyretics, antirheumatic agents, antiseptics, antithyroid agents, antiulcer agents, antiviral agents, anxiolytic sedatives, astringents, beta-adrenoceptor blocking agents, biocides, blood cloning factors, calcitonin, cardiotonics, chemotherapeutics, cholesterol lowering agents, corticosteroids, cough suppressants, cytokines, diuretics, dopaminergics, estrogen receptor modulators, enzymes, enzyme cofactors, enzyme inhibitors, growth differentiation factors, growth factors, hematological agents, hematopoietics, hemoglobin modifiers, lemostatics, hormones and hormone analogs, hypnotics, hypotensive diuretics, immunological agents, immunostimulants, immunosuppressants, inhibitors, ligands, lipid regulating agents, lymphokines, muscarinics, muscle relaxants, neural blocking agents, neurotropic agents, paclitaxel and derivative compounds, parasympathomimetics, parathyroid hormone, promoters, prostaglandins, psychotherapeutic agents, psychotropic agents, radio-pharmaceuticals, receptors, sedatives, sex hormones, sterilants, stimulants, thrombopoietics, trophic factors, sympathomimetics, thyroid agents, vaccines, vasodilators, vitamins, xanthines, as well as conjugates, complexes, precursors, and metabolites thereof. The active agent may be used individually or in combinations of two or more thereof. In one example, the active agent is a prophylactic and/or therapeutic agent that includes, but is not limited to, peptides, proteins, carbohydrates, polysaccharides, nucleic acids, nucleotides, other compounds, precursors and derivatives thereof, and combinations of two or more thereof. The active agent includes, without limitation, vectors such as viruses, and virus particles. In one example, the active agent is negatively charged. The active agent further includes a nucleic acid, DNA, RNA, a plasmid, a viral vector, an oligonucleotide, an antisense nucleic acid, a missense nucleic acid, or a mixture thereof.

In one example, the active agent is a cosmetic agent. Non-limiting cosmetic agents include inter-alia emollients, humectants, free radical inhibitors, anti-inflammatories, vitamins, depigmenting agents, anti-acne agents, antiseborrhoeics, keratolytics, slimming agents, skin coloring agents, and sunscreen agents. Non-limiting compounds useful as cosmetic agents include linoleic acid, retinol, retinoic acid, ascorbic acid alkyl esters, polyunsaturated fatty acids, nicotinic esters, tocopherol nicotinate, unsaponifiables of rice, unsaponifiables of soybean, unsaponifiables of shea, ceramides, hydroxy acids such as glycolic acid, selenium derivatives, antioxidants, beta-carotene, gamma-orizanol, and stearyl glycerate. The cosmetic agents may be commercially available and/or prepared by known techniques.

In one example, the active agent is a nutritional supplement. Non-limiting nutritional supplements include proteins, carbohydrates, water-soluble vitamins (e.g., vitamin C, B-complex vitamins, and the like), fat-soluble vitamins (e.g., vitamins A, D, E, K, and the like), and herbal extracts. The nutritional supplements may be commercially available and/or prepared by known techniques.

In one example, the active agent is a compound having a molecular weight of 2 kD or less. Non-limiting examples of such compounds include steroids, beta-agonists, anti-microbials, antifungals, taxanes (antimitotic and antimicrotubule agents), amino acids, aliphatic compounds, aromatic compounds, and urea compounds.

In one example, the active agent may be a therapeutic agent for prevention and/or treatment of pulmonary disorders. Non-limiting examples of such agents include steroids, beta-agonists, anti-fungals, anti-microbial compounds, bronchial dialators, anti-asthmatic agents, non-steroidal anti-inflammatory agents (NSAIDS), AAT, and agents to treat cystic fibrosis. Non-limiting examples of steroids include beclomethasone (such as beclomethasone dipropionate), fluticasone (such as fluticasone propionate), budesonide, estradiol, fludrocortisone, flucinonide, triamcinolone (such as triamcinolone acetonide), flunisolide, and salts thereof. Non-limiting examples of beta-agonists include salmeterol xinafoate, formoterol fumarate, levo-albuterol, bambuterol, tulobuterol, and salts thereof. Non-limiting examples of anti-fungal agents include itraconazole, fluconazole, amphotericin B, and salts thereof.

In one example, the active agent may be a diagnostic agent. Non-limiting diagnostic agents include x-ray imaging agents and contrast media. Non-limiting examples of x-ray imaging agents include ethyl 3,5-diacetamido-2,4,6-triiodobenzoate (WIN-8883, ethyl ester of diatrazoic acid); 6-ethoxy-6-oxohexyl-3,5-bis(acetamido)-2,4,6-triiodobenzoate (WIN 67722); ethyl-2-(3,5-bis(acetamido)-2,4,6-triiodobenzoyloxy)-butyrate (WIN 16318); ethyl diatrizoxyacetate (WIN 12901); ethyl 2-(3,5-bis(acetamido)-7,4,6-triiodobenzoyloxy)propionate (WIN 16923); N-ethyl 2-(3,5-bis(acetamido)-2,4,6-triiodobenzoyloxy-acetamide (WIN 65312); isopropyl 2-(3,5-bis(acetamide)-2,4,6-triiodobenzoyloxy)acetamide (WIN 12855); diethyl 2-(3,5-bis(acetamido)-2,4,6-triiodobenzoyloxymalonate (WIN 67721); ethyl 2-(3,5-bis(acetamido)-2,4,6-triiodobenzoyloxy)phenyl-acetate (WIN 67585); propanedioic acid, [[3,5-bis(acetylamino)-2,4,5-triodobenzoyl]oxy]bis(1-methyl)ester (WIN 68165); and benzoic acid, 3,5-bis(acetylamino)-2,4,6-triodo-4-(ethyl-3-ethoxy-2-butenoate)ester (WIN 68209). Preferred contrast agents desirably disintegrate relatively rapidly under physiological conditions, thus minimizing any particle associated inflammatory response. Disintegration may result from enzymatic hydrolysis, solubilization of carboxylic acids at physiological pH, or other mechanisms. Thus, poorly soluble iodinated carboxylic acids such as iodipamide, diatrizoic acid, and metrizoic acid, along with hydrolytically labile iodinated species such as WIN 67721, WIN 12901, WIN 68165, and WIN 68209 or others may be preferred.

The active agents may be used in a combination of two or more thereof. Non-limiting examples include a steroid and a beta-agonist, e.g., fluticasone propionate and salmeterol, budesonide and formoterol, etc.

In one example, the microparticles are substantially free of internal voids and/or cavities (such as being free of vesicles), substantially free of encapsulation, substantially free of lipids, substantially free of hydrogel, substantially a non-porous, amorphous solid, and/or substantially spherical as those terms are defined herein. The microparticles may have multiple surface channel openings, the diameter of which are generally 100 nm or less, preferably 10 nm or less, more preferably 5 nm or less, most preferably 1 nm or less. Microparticle cores may have an overall density of 0.5 g/cm³ or greater, preferably 0.75 g/cm³ or greater, more preferably 0.85 g/cm³ or greater. The density may be generally up to about 2 g/cm³, preferably 1.75 g/cm³ or less, more preferably 1.5 g/cm³ or less.

In one example, the microparticles exhibit a high payload of the at least one active agent. Depending on the formulation and the physical/chemical nature of the compounds, there are typically at least 1000 or more, such as a few million to hundreds of millions of the active agent molecules in each of the microparticles. The weight percentage of the active agent in the microparticle may be any of the amount below or greater, or any ranges there between, but less than 100%: 10%, 15%, 20%, 25%, 30%, 35%, 40%, 45%, 50%, 55%, 60%, 65%, 70%, 75%, 80%, 85%, 90%, 95%, 96%, 97%, 98%, 99%. While incorporation of a significant amount of bulking agents and/or other excipients is not required in the microparticles, one or more of such compounds may be present therein. In any event, the desired integrity and/or activity are retained for a majority (for example, 50% or greater, preferably 75% or greater, more preferably 90% or greater, most preferably 95% or greater) of the active agent molecules, if not 100%.

In one example, the microparticles may have a weight percent (wt. %) loading of the active agent of 40% or more, preferably 60% or more, or 80% or more, or 90% or more, or 95% or more, and less than 100%, typically 98% or less. The microparticles may further have, or are capable of being induced (such as from a neutral state) to have, a net surface electric charge. In one example, the net surface charge is contributed primarily or essentially by the active agent and/or the carrier, if any, present in the microparticles; the compound(s) may preferably be homogeneously distributed therein. Alternatively, the active agent is compartmentalized in one or more portions of the microparticle, such as a center, or is preferably distributed substantially homogeneously within the portion.

In one example, each of the surface-modified microparticles of the present disclosure preferably contains an amorphous (e.g., such as free of three-dimensional crystalline structures) and solid microparticle core associated with at least one monolayer containing at least one charged compound. The microparticle contains at least one active agent, for example, at least one macromolecule. The macromolecule can have a molecular weight of 4,500 Daltons or greater, and can be negatively charged, can be positively charged, or can have an inducible charge. The macromolecule may be the active agent or may be different from the active agent. The macromolecule can include carbohydrates (e.g., oligosaccharides, polysaccharides), peptides, proteins (e.g., enzymes, recombinant proteins, albumins, monoclonal antibodies, polyclonal antibodies, glycoproteins, and fragments and derivatives thereof), viruses (e.g., viral particles), lipids, nucleic acids (e.g., sense, anti-sense, or missense DNA or RNA oligonucleotides; aptamers; sense, anti-sense, or missense DNA or RNA polynucleotides; siRNA; nucleotide acid constructs; nucleotides; vectors; viral vectors; and fragments and derivatives thereof), and complexes and conjugates thereof. The macromolecule can be a monoclonal antibody, a polyclonal antibody, aantnticancer agent, an anticoagulant, an antigen, an anti-inflammatory agent, a blood clotting factor, a cytokine, an enzyme, an enzyme cofactor, an enzyme inhibitor, a growth differentiation factor, a growth factor, an immunological agent, a parathyroid hormone, a vaccine, and mixtures thereof. The macromolecule may be a carrier, a stabilizing agent, or a complexing agent (e.g., proteinaceous compounds, polyelectrolytes). The active agent and/or the macromolecule may constitute 40% to 100% or less, and typically at least 80%, such as 90% or more, or 95% or more, by weight of the microparticle core. Preferably, the active agent and/or the macromolecule is/are distributed homogeneously throughout the microparticle core. An outer surface of the microparticle core carries a net surface charge, which may be attributed, at least in part, and more typically in large part, to the active agent and/or the macromolecule, especially when the outer surface is formed of the active agent and/or the macromolecule. The microparticle core may be free of covalent crosslinking, hydrogel, lipids, and/or encapsulation. Alternatively, the microparticle core may contain one or more charged compounds, covalent crosslinking, and/or encapsulation. The one or more charged compounds in the microparticle core may be distributed homogeneously throughout the microparticle core, or compartmentalized in specific portions thereof, such as in a layer. The microparticle may preferably have a particle size of 50 μm or less, and may have a monodisperse or polydisperse size distribution.

### Surface-Modified Microparticles

Surface modification of the microparticles is achieved, without limitation, by forming, in a controlled manner, at least one monolayer (such as a coating) containing at least one ionic polymer and/or at least one grafted polymer as described herein, i.e., a polymer comprising an amphiphilic polymer or nonionic polymer grafted to an ionic polymer, about the microparticle core. Scheme 1 shows an example of a grafted polymer having polyethylene glycol as the amphiphilic polymer or nonionic polymer grafted to polylysine as the ionic polymer.

### Scheme 1

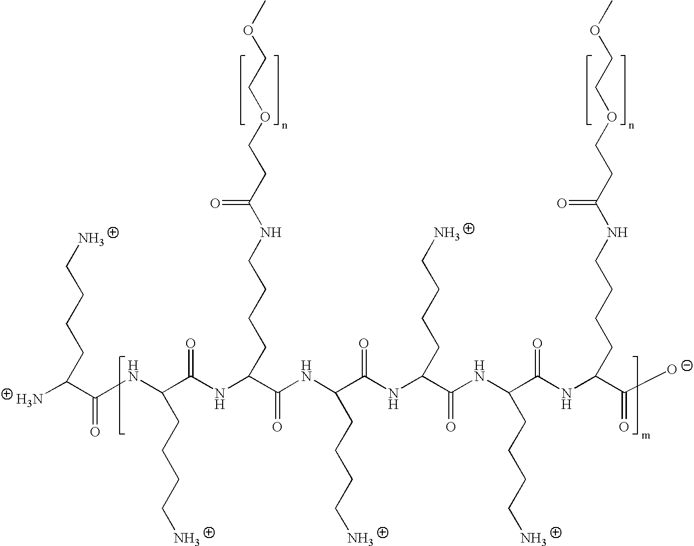

The microparticles may be exposed to (such as mixed with) at least one charged compound and/or a grafted polymer having or capable of having a net electrical charge that is, preferably, opposite in sign to the net surface charge of the microparticle, thereby forming the formed monolayer of the charged compound and/or a grafted polymer about the microparticle. The formed monolayer of the surface-modified microparticle has a net surface electric charge that may be the same in sign as that of the microparticle core, zero or, preferably, opposite in sign to that of the microparticle core. In other words, if the outer surface of the microparticle core has a negative net surface charge (such as determined by zeta-potential measurements), and only a single monolayer is included, then the formed monolayer may preferably have on its outer surface a positive net surface charge. Alternatively, if the microparticle core has a positive net surface charge, and only a single monolayer is included, then the formed monolayer may preferably have a negative net surface charge. Deposition of the monolayer can take place in an aqueous medium (e.g., water, buffer, or aqueous solution containing some water miscible organic solvent of the type previously described, or one that may be present in the manufacture of the microparticle core). In microparticles comprising more than one monolayer, typically only the outermost layer includes a grafted polymer as described herein (see, for example, FIG. 8). When two or more distinct monolayers are formed about the microparticle core, each typically contains different charged species, and preferably each carries on its outer surface a non-zero net surface charge that is different in sign and/or value from that of the preceding one and/or the subsequent one, if present. The microparticle core can similarly carry a non-zero net surface charge, e.g., the core can be positively charged, negatively charged, or neutral in charge. When the microparticle core has a positive charge or a negative charge, the monolayer immediately adjacent to the microparticle core can have a charge that is opposite in sign to the charge of the microparticle core. When the microparticle core is uncharged, the monolayer immediately adjacent to the microparticle core can be positively charged or the monolayer can be negatively charged. Deposition of the monolayers one at a time allows for optimal control over various properties of the resulting microparticles, allowing one to tailor or “fine-tune” the microparticles to achieve a desired result. In each instance, at least the outermost layer is a layer comprising an amphiphilic or a nonionic polymer grafted to an ionic polymer, as disclosed herein. Preferably, the monolayer immediately about the microparticle (“formed monolayer”) contains one or more charged compounds, each independently having a net charge that is opposite in sign to the net surface charge of the microparticle core. The microparticle may at least, in part, be penetrable by the charged compound in the formed monolayer. An outer surface of the formed monolayer may carry a net surface charge that is different from, preferably opposite in sign to, that of the microparticle outer surface, especially when the formed monolayer is a saturated monolayer as defined herein. The charged compounds may include one or more of ionic polymers such as polyelectrolytes, charged polyaminoacids, charged polysaccharides, polyionic polymers, charged proteinaceous compounds, and/or charged peptides, or other charged compounds such as charged lipids optionally in combination with uncharged lipids, charged lipid structures, and derivatives thereof.

The surface-modified microparticle may further contain one or more additional monolayers of alternating charge, such that the surface-modified microparticle has a desired release profile of the active agent. This number is not particularly limited, but may typically be between 1 to 20, such as 2, 3, 4, 5, 6, 7, 8, 9, 10, 11, 12, 13, 14, 15, 16, 17, 18, 19, or 20. Optionally, one or more of such charged monolayers may independently have one or more active agents, such as an affinity molecule, especially a targeting ligand, associated covalently and/or non-covalently thereto, preferably on their respective outer surfaces. Alternatively or in combination, the microparticle core may have one or more portions, such as a center or an underlying layer (a charged monolayer, for example), containing at least one such active agent, preferably on the outer surface of the portion. In each instance, at least the outermost layer is a layer comprising an amphiphilic or a nonionic polymer grafted to an ionic polymer, as disclosed herein.

The microparticle, the surface-modified microparticle, and any intermediates there between, if any, may be and/or may have one or more of the following characteristics: spherical as defined herein, free of covalent crosslinking, free of hydrogel and/or swelling, and have a polydisperse or, preferably, monodisperse, size distribution. The microparticle may be free of lipids and/or encapsulation.

Preferably, the surface-modified microparticle is capable of controlled release, especially sustained release, of the active agent, with a non-limiting release profile such as an initial burst and a linear release profile, and may be provided as a suspension or a dry powder in compositions or formulations for pharmaceutical, therapeutic, diagnostic, cosmetic, and/or nutritional applications. The controlled release may occur within a selected pH environment. In that regard, preferably the controlled release may occur within a pH range of approximately 2 to 10, and more preferably approximately 5 to 7.5, such as a physiological pH of 7 to 7.4 or an endosomal pH of 5 to 6.5.

Controlled deposition of the one or more monolayers may further involve alteration of the net surface charge of the microparticle core (or of a microparticle having one or more monolayers associated therewith) through a controlled manipulation of one or more conditions, such as changes in temperature, pressure, pH, ionic strength and/or osmolality of the reaction medium, concentrations of components within the reaction medium, the likes thereof, as well as rates of such changes, and combinations of two or more thereof. Such controlled manipulations may desirably be applied before and up to the deposition of the one or more monolayers, or even during the monolayer formations. In one example, the net surface charge of the microparticle is capable of being positive, neutral, or negative. The net surface charge is selected through, for example, a controlled change in one or more of the conditions described above, such as a controlled change in pH. In one example, the pH of the solution is selected such that the net surface charge of the microparticle is negative, and the difference between the pH of the solution and the surface-neutral point of the microparticle (i.e., the pH at which the microparticle is neutral) is less than 0.3, alternatively equal to or greater than 0.3, preferably 0.5 or greater, more preferably 0.8 or greater, most preferably 1 or greater.

The surface-modified microparticle can further comprise a second monolayer, and the second monolayer can be between the monolayer comprising the amphiphilic polymer or nonionic polymer grafted to the ionic polymer and an outer surface of the microparticle core. The monolayer comprising the amphiphilic polymer or nonionic polymer grafted to the ionic polymer can be adjacent to the second monolayer, and the second monolayer can carry a net charge that is opposite in sign to the net charge of the monolayer comprising the amphiphilic polymer or nonionic polymer grafted to the ionic polymer.

The surface-modified microparticle also can comprise at least first and second monolayers and the net charge of the first monolayer can be opposite in sign to the net charge of the second monolayer. The microparticle core can further comprise an outer surface carrying a net surface charge, and the layer comprising the amphiphilic polymer or nonionic polymer grafted to the ionic polymer can carry a net charge that is the same in sign as the net charge of the microparticle core.

The monolayer associated immediately about the microparticle core generally has a charge that is opposite in sign to the charge of the microparticle core. If the microparticle core is uncharged, the monolayer associated immediately about the microparticle core may be positively charged or negatively charged. The surface-modified microparticles may comprise more than one monolayer, and adjacent monolayers usually have charges that are opposite in sign from that of the preceding one and/or the subsequent one, if present. At least one monolayer comprises an amphiphilic polymer or a non-ionic polymer grafted to an ionic polymer. At least one monolayer comprising a grafted polymer as described herein provides the outermost layer of the microparticle.

The microparticle cores, the surface-modified microparticles, and any intermediates there between, generally have an average particle size from about 0.01 μm to about 1000 μm, for example, from about 0.01 μm to about 500 μm, from about 0.01 μm to about 200 μm, from about 0.1 μm to about 100 μm, from about 0.1 μm to about 50 μm, from about 0.1 μm to about 10 μm, from about 0.1 μm to about 5 μm, from about 0.1 μm to about 2 μm, and/or from about 0.1 μm to about 1 μm.

Preferably, the microparticles, the surface-modified microparticles, and any intermediates there between, have a narrow particle size distribution. What is meant by a narrow particle size distribution is that the ratio of the volume diameter of the 90th percentile (i.e., the average particle size of the largest 10% of the microparticles) to the volume diameter of the 10th percentile (i.e., the average particle size of the smallest 10% of the microparticles) is about 5 or less, about 4 or less, about 3 or less, about 2 or less, and about 1.5 to 1. A Geometric Standard Deviation (GSD) of 2.5 or less, preferably 1.8 or less, may also be used to indicate a monodisperse size distribution. Calculation of GSD is known and understood to one skilled in the art.

The microparticle cores, the surface-modified microparticles, and any intermediates there between, generally have an overall density of about 0.5 g/cm³ to about 2 g/cm³, for example, about 0.6 g/cm³ to about 1.9 g/cm³, about 0.7 g/cm³ to about 1.8 g/cm³, about 0.75 g/cm³ to about 1.75 g/cm³, about 0.8 g/cm³ to about 1.7 g/cm³, about 0.8 g/cm³ to about 1.65 g/cm³, about 0.8 g/cm³ to about 1.6 g/cm³, about 0.8 g/cm³ to about 1.55 g/cm³, about 0.85 g/cm³ to about 1.5 g/cm³, about 0.9 g/cm³ to about 1.45 g/cm³, about 0.95 g/cm³ to about 1.4 g/cm³, about 1 g/cm³ to about 1.35 g/cm³, about 1.05 g/cm³ to about 1.3 g/cm³, about 1.1 g/cm³ to about 1.25 g/cm³, and/or about 1.15 g/cm³ to about 1.2 g/cm³.

### Polymer-Grafted Ionic Polymers

At least the outermost layer of the surface-modified microparticles comprises an amphiphilic polymer or a non-ionic polymer grafted to an ionic polymer. The ionic polymer comprises one or more types of monomer unit, and at least one monomer type comprises one or more modifiable functional groups. As used herein, a “modifiable functional group” is a chemical group having a known reactivity with a second chemical group or groups such that when the modifiable functional group is exposed to a compound comprising the second chemical group under specific conditions (e.g., in the presence of an activating reagent), a covalent bond or association is formed between the modifiable functional group and the second chemical group. Examples of modifiable functional groups of such ionic polymers include, but are not limited to, an amino group, a carboxyl group, a thiol group, a hydroxyl group, an epoxy group, a haloalkyl group (e.g., a chloroethyl group, or a chloropropyl group), an aldehyde group, a carbonyl group, an isocyanate group, an imino group, and a nitrile (also known as cyano) group. Examples of suitable second chemical groups for reacting and forming covalent associations with said modifiable functional groups include, but are not limited to, N-hydroxy succinimide esters, amines, tresylates, aldehydes, epoxides, p-nitrophenyl carbonates, cyanuric chlorides, isocyanates, carbonyl imidazoles, vinyl sulfones, maleimides, dithioorthopyridines, and derivatives thereof. For example, the amino group of lysine residues in proteins and peptides can react with N-hydroxy succinimide esters to form modified lysine residues, and the carboxylate groups of glutamate and aspartate residues can react with amines in the presence of activating reagents (e.g., N,N′-dicyclohexylcarbodiimide (DCC), N,N′-diisopropylcarbodiimide (DIC), and 1-ethyl-3-[3-dimethylaminopropyl]carbodiimide hydrochloride (EDC or EDAC)) to form modified glutamate and aspartate residues.

The degree of grafting of the amphiphilic polymer or non-ionic polymer to the ionic polymer is defined as moles of amphiphilic polymer or non-ionic polymer divided by moles of modifiable functional groups of the ionic polymer. Typically, the ionic polymer will comprise one modifiable functional group per monomer, and therefore the moles of modifiable functional groups will equal the moles of monomer, but the ionic polymer could comprise more than one modifiable functional group per monomer. In various embodiments, the degree of grafting of the amphiphilic polymer or non-ionic polymer to the ionic polymer can be about 1% to about 30%, for example, about 5% to about 25%. The degree of grafting also can be about 5% to about 20%, about 5% to about 15%, and about 5% to about 10%.

The amphiphilic polymer or non-ionic polymer can be selected from a variety of known amphiphilic polymers or non-ionic polymers. Suitable amphiphilic polymers or non-ionic polymers include, but are not limited to polyethylene glycols, branched polyethylene glycols, polyimides, polyesters, polyethers, polypropylene glycols, aryl alkyl polyether alcohols, polyaliphatic alcohols, polyethylene glycol acrylates, polyvinyl polymers, polyaldehydes, polyoxyethylene fatty alcohol ethers (e.g., MACROGOL™ and BRIJ™), polyoxyethylene sorbitan fatty acid esters (i.e., polysorbates), polyoxyethylene fatty acid esters (e.g., MYRJ™), sorbitan esters (e.g., SPAN™), naturally occurring polymers, and mixtures thereof. Specific amphiphilic polymers or non-ionic polymer include, but are not limited to, polyethylene oxide, polyethylene imine, polyvinyl acetate, polyvinyl alcohol, polyvinylpyrrolidone, zein, poly[N-tris(hydroxymethyl)methylmethacrylate], polyoxyethylene sorbitan, poly(ethylene glycol)(n)monomethyl ether mono(succinimidylsuccinate)ester, poly(perfluoropropylene oxide-b-perfluoroformaldehyde), poly(tetramethylene ether glycol), and mixtures thereof. Co-polymers, block co-polymers, ter-polymers, branched polymers, and/or cyclo-polymers of any of the foregoing amphiphilic polymers or non-ionic polymers also are suitable, for example, polyoxyethylene-polyoxypropylene copolymers (poloxamers) such as poloxamer 407 and PLURONIC L-101™ polymer, and poloxamines. Additional suitable amphiphilic polymers or non-ionic polymers include, but are not limited to carbohydrate-based polymers (polysaccharides) such as carboxymethyl cellulose-based polymers, cyclodextrins, methylcellulose, dextran, polydextrose, chitin, chitosan, hydroxymethylcellulose, hydroxypropylcellulose, hydroxypropylmethylcellulose, noncrystalline cellulose, pullulan, starch, starch derivatives (i.e., hydroxyethylstarch (HES) and hetastarch), and mixtures and derivatives thereof.

In one embodiment, the amphiphilic polymer or non-ionic polymer can have a molecular weight of from about 100 Da to about 30,000 Da, for example, from about 200 Da to about 25,000 Da, from about 500 Da to about 20,000 Da, from about 750 Da to about 15,000 Da, from about 900 Da to about 10,000 Da, from about 1,000 Da to about 5,000 Da, and/or from about 1,500 Da to about 2,500 Da. In one aspect according to this embodiment, the amphiphilic or nonionic polymer comprises polyethylene glycol.

The ionic polymer may be selected from polyelectrolytes, charged polyaminoacids, charged polysaccharides, charged proteinaceous compounds, and/or charged peptides. Suitable ionic polymers are disclosed below. The ionic polymer can be positively charged (cationic) or negatively charged (anionic). Examples of positively charged polymers include, but are not limited to, polyamino acids such as polylysine, polyhistidine, polyornithine, polycitrulline, polyhydroxylysine, polyarginine, polyhomoarginine, polyaminotyrosine, and protamines. Other suitable positively charged polymers include, but are not limited to, polydiaminobutyric acid, polyethyleneimine, polypropyleneimine, polyamino(meth)acrylate, polyaminostyrene, polyaminoethylene, poly(aminoethyl)ethylene, polyaminoethylstyrene, diethyl amino ethyl cellulose, poly-imino tyrosine, cholestyramine-resin, poly-imino acid, 1,5-dimethyl-1,5-diazaundecamethylene polymethobromide (hexadimethrine bromide), chitosan, poly(amidoamine) dendrimers, and combinations thereof. Cationic polymers of the present disclosure comprise one or more positively charged monomers, and examples of positively charged monomers include, but are not limited to, lysine, histidine, ornithine, hydroxylysine, arginine, homoarginine, aminotyrosine, diaminobutyric acid, ethyleneimine, propylenimine, amino(meth)acrylate, aminostyrene, aminoethylene, aminoethylethylene, aminoethylstyrene, citrulline, diethyl amino ethyl glucose, imino tyrosine, (vinylbenzyl)trimethylammonium salts, imino acids, quaternary alkyl ammonium salts, amidoamines, glucosamine, and mixtures and derivatives thereof. Examples of negatively charged polymers include, but are not limited to, polyaspartic acid, polyglutamic acid, polyacrylic acid, polymethacrylic acid, polymaleic acid, polymaleic acid monoester, heparin sulfate, dextran sulfate, polygalacturonic acid, polyalginate(polyaginic acid), polypectimic acid, polymannuronic acid, polyguluronic acid, polysialic acid, polycarboxymethyl cellulose, polyhyaluronic acid, chondroitin sulfate, chitosan sulfate, glycosaminoglycans, proteoglycans, and mixtures thereof. Anionic polymers of the present disclosure comprise one or more negatively charged monomers, and examples of negatively charged monomers include, but are not limited to, aspartic acid, glutamic acid, acrylic acid, methacrylic acid, maleic acid, maleic acid monoester, heparin sulfate, dextran sulfate, galacturonic acid, alginate (aginic acid), pectimic acid, mannuronic acid, guluronic acid, sialic acid, carboxymethyl glucose, hyaluronic acid, chondroitin sulfate, sulfated glucose, sulfated glucuronic acid, sulfated iduronic acid, sulfated glucosamine, sulfated acetylgalactosamine, glycosaminoglycan-modified amino acids, sulfated carbohydrates, and mixtures thereof. Both D- and L-optical isomers of the charged polymers are encompassed by the present disclosure.

The concentration of the grafted polymer when mixed with the microparticle core to form the surface-modified microparticles is typically from about 0.01 mg/mL to about 10 mg mL, for example, about 0.1 mg/mL to about 5 mg/mL, about 0.5 mg/mL to about 3 mg/mL, and/or about 1 mg/mL to about 2 mg/mL.

Beneficially, the surface-modified microparticles of the present disclosure, when administered to a subject, can demonstrate altered cell uptake compared to the cell uptake of a corresponding unmodified microparticle core. For example, the surface-modified microparticle can demonstrate at least 50% less cell uptake compared to the cell uptake of the unmodified microparticle core. In additional examples, the surface-modified microparticle demonstrates at least 55%, at least 60%, at least 65%, at least 70%, at least 75%, at least 80%, and/or at least 85% less cell uptake compared to the cell uptake of the unmodified microparticle core. The surface-modified microparticles of the present disclosure, when administered to a subject, also demonstrate altered cell uptake compared to the cell uptake of surface-modified microparticles coated with the non-grafted ionic polymer. For example, the surface-modified microparticle demonstrates at least 50% less cell uptake compared to the cell uptake of surface-modified microparticles coated with the non-grafted ionic polymer. Inadditional examples, the surface-modified microparticle demonstrates at least 55%, at least 60%, at least 65%, at least 70%, at least 75%, at least 80%, and/or at least 85% less cell uptake compared to the cell uptake of surface-modified microparticles coated with the non-grafted ionic polymer.

Grafting of the amphiphilic polymer or non-ionic polymer to the ionic polymer provides a monolayer coating for the microparticles that can improve the stealth properties of the microparticles within a mammalian subject. As used herein, “stealth” refers to the reduced recognition and uptake of the microparticles by the various phagocytic and other cell types that function to accelerate the clearance of the microparticles from the body. Various amphiphilic polymers or non-ionic polymers can provide stealth properties to the microparticles of the present disclosure, including, but not limited to polyethylene glycols, poloxamers, carbohydrate-based polymers (e.g., hydroxyethyl starch polymers, polysialic acid, carboxymethyl cellulose-based polymers, cyclodextrins), polyaliphatic alcohols, polyethylene glycol acrylates, poly(vinyl)polymers, polyethers, polyimides, polyesters, polyaldehydes, copolymers (e.g., terpolymers, block copolymers) thereof, and mixtures and derivatives thereof. Thus, glycosylation and sialylation of suitable amphiphilic and nonionic polymers can improve the stealth properties of the microparticles. Substantially no covalent bonds are present between the macromolecule of the microparticle core and the amphiphilic polymer or nonionic polymer.

### Pharmaceutical Compositions

The present disclosure is directed to a pharmaceutical composition comprising a plurality of surface-modified microparticles. Generally, the pharmaceutical composition further comprises one or more excipients, also referred to as inactive ingredients. Excipients can be added to the pharmaceutical composition of the present disclosure to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., the U.S. Pharmacopoeia (USP), Japanese Pharmacopoeia (JP), European Pharmacopoeia (EP), and British pharmacopeia (BP); the U.S. Food and Drug Administration Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.). Non limiting examples of suitable excipients include maize starch, wheat starch, rice starch, potato starch, glucose, lactose, sucrose, mannitol, sorbitol, gelatin, gum, methyl cellulose, hydroxypropylmethyl-cellulose, sodium carboxymethylcellulose, polyvinylpyrrolidone (PVP), crosslinked polyvinyl pyrrolidone, agar, alginic acid, sodium alginate, malt, rice, flour, chalk, silica gel, sodium stearate, glycerol monostearate, talc, sodium chloride, calcium phosphate, tragacanth, calcium silicate, magnesium stearate, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

In one aspect, the pharmaceutical composition is suitable for pulmonary administration. Examples of suitable microparticle compositions for pulmonary administration are described in U.S. Patent Publication No. 2007/0092452. Particles for pulmonary administration may be deposited to the deep lung, in the upper respiratory tract, or anywhere in the respiratory tract. The particles may be delivered as a dry powder by a dry powder inhaler, or they may be delivered by a metered dose inhaler or a nebulizer. Drugs intended to function systemically, are desirably deposited in the alveoli, where there is a very large surface area available for absorption into the bloodstream. When targeting the drug deposition to certain regions within the lung, the aerodynamic diameter of the particle can be adjusted to an optimal range by manipulating fundamental physical characteristics of the particles such as shape, density, and particle size. Acceptable fractions of inhaled drug particles are often achieved by adding excipients to the formulation, either incorporated into the particle composition or as a mixture with the drug particles. In order to minimize potential deleterious side effects of deep lung inhaled therapeutics, it may be advantageous to fabricate particles for inhalation that are substantially constituted by the drug to be delivered. The requirements to deliver particles to the deep lung by inhalation are that the particles have a small mean aerodynamic diameter of 0.5-10 μm and a narrow size distribution. An alternative approach is to produce particles with relatively low porosity, wherein the particles have a density, generally that is close to 1 g/cm3.

In another embodiment, the pharmaceutical composition is suitable for injectable administration. For example, the microparticles can be administered intravenously, intraarterially, intramuscularly, subcutaneously, intradermally, intraarticularly, intrathecally, epidurally, intracerebrally, and intraperitoneally.

### Methods of Preparing Surface-Modified Microparticles Having a Monolayer Comprising an Amphiphilic Polymer or a Nonionic Polymer Grafted to an Ionic Polymer

The present disclosure is directed to a process for preparing surface-modified microparticles comprising a grafted polymer, the process comprising (a) providing a microparticle core comprising a macromolecule typically being selected from the group consisting of carbohydrates, peptides, proteins, vectors, nucleic acids, complexes thereof, and conjugates thereof; (b) admixing an activated amphiphilic polymer or non-ionic polymer and an ionic polymer under conditions sufficient to form a grafted polymer comprising the amphiphilic polymer or non-ionic polymer grafted to the ionic polymer; and (c) admixing the grafted polymer of (b) for example, at a concentration of about 0.01 mg/mL to about 2 mg/mL, and the microparticle core under conditions sufficient to form a surface-modified microparticle comprising the grafted polymer and having an outermost monolayer, said outermost monolayer comprising the amphiphilic polymer or nonionic polymer grafted to the ionic polymer. In one embodiment, the microparticle core can be provided as a surface-modified microparticle (which itself necessarily comprises a microparticle core).

Other macromolecules may also be used in the disclosed methods. Further still, microparticle cores comprising active agents other than macromolecules may be used in the disclosed methods. For example, microparticle cores comprising active agents having reactive (or modifiable functional) groups, particularly small molecules having modifiable functional groups, may be used in the disclosed methods.

Microparticles prepared in accordance with the present disclosure have at least one monolayer comprising an amphiphilic polymer or a non-ionic polymer grafted to an ionic polymer. Because grafting of the amphiphilic polymer or non-ionic polymer to the ionic polymer is carried out prior to coating the microparticles with the grafted ionic polymer, the active agent(s) of the microparticle core are not covalently modified by the activated amphiphilic polymer or non-ionic polymer. This can be advantageous, as covalent modification of the agent(s) comprising the microparticle core may reduce the activity and pharmacokinetics/pharmacodynamics of the active agent(s) particularly if the active agent comprises modifiable functional groups (for example, macromolecule active agents such as proteins, nucleic acids, carbohydrates, and the like). Similarly, many small molecule active agents can possess modifiable functional groups that advantageously remain unmodified by the amphiphilic polymer or non-ionic polymer during the coating steps.

Often, the microparticles have more than one monolayer, which are distinct from one another. To prepare a microparticle having one or more charged monolayers associated therewith, a non-limiting method includes pre-forming or otherwise providing an unmodified microparticle, exposing it to the one or more grafted polymer or charged compounds, which may be provided in a solution into which the microparticle may be immersed, and forming the monolayer. The solution may contain one or more of water, a buffer, and a water-miscible organic solvent, and one or more solubility reducing agents (e.g., alcohols, carbohydrates, non-ionic aqueous-miscible polymers, and/or inorganic ionic compounds containing monovalent or multivalent cations), with a concentration in weight-to-volume percentage of 5% to 50%, preferably 10% to 90%. A non-limiting example of the solution contains about 16% (w/v) polyethylene glycol and 0.7% (w/v) NaCl. The pH of the solution, typically in a range of 4 to 10, may be adjusted to be same as or close to the surface-neutral point of the microparticle core (such as with a difference of 0 to less than 0.3), or away from that (such as with a difference of 0.3 pH units or greater). The grafted polymer or charged compound may be present in the solution at a concentration of 0.05 mg/mL to 10 mg/mL. The microparticle core and the grafted polymer or charged compound are co-incubated in the solution, preferably at a temperature of 2° C. to 5° C. or up to ambient temperature over a period of 1 second to 10 hours. The formation of the monolayer may be carried out in a controlled manner. The resulting surface-modified microparticle or an intermediate thereof may be separated from the solution with optional washing. The washing medium may be the same as the solution described above. The procedure may be repeated using alternatingly charged grafted polymers and/or compounds to form the alternatingly charged monolayers, if desired. The monolayer comprising the grafted polymer typically is the outermost monolayer.

As indicated above, the reaction system can include one or more solubility reducing and/or viscosity increasing agents (SRA/VIA), as well as one or more PSEAs. Suitable SRA/VIAs and PSEAs include, without limitation, those known to one skilled in the art and those disclosed herein, such as alcohols (e.g., ethanol, glycerol), carbohydrates (such as sucrose), non-ionic aqueous-miscible polymers (e.g., PEG, PVP, block copolymers of polyoxyethylene and polyoxypropylene(poloxamers), hetastarch, dextran, etc.), and inorganic ionizable compounds containing multivalent (e.g., divalent, trivalent) cations (e.g., metal and organic cations such as those disclosed herein), such as ZnCl₂.

Thus, in one example, deposition of the formed monolayer may take place in a solution that includes buffered saline (that is, 0.7% NaCl buffer) and 8% or more by weight or volume of a SRA/VIA such as PEG, preferably 12% or more, more preferably 15% or more; typically 30% or less, preferably 25% or less, more preferably 20% or less, most preferably about 16% or more. The amount of SRA/VIA required in the solution will depend, in part, on the stability of the active agent, as well as the dissolution profile of the monolayer(s). Certain grafted polymers and charged compounds (such as the polycations gelatin B and chitosan) may work in solutions containing 16% or less SRA/VIA.

The pH of the solution at which the net surface charge of the microparticle is zero is referred herein as the surface-neutral point of the microparticle in the particular solution. In certain examples, the pH of the solution may be adjusted to be at or near, the surface-neutral point of the microparticle in the solution with a difference there between of less than 0.3 (pH units), preferably 0.25 or less, more preferably 0.2 or less. In other examples, the pH of the solution may preferably be adjusted away from the surface-neutral point of the microparticle in the solution, with a difference there between of 0.3 (pH units) or greater, preferably 0.5 or greater, more preferably 0.8 or greater, most preferably 1 or greater. It has been observed that in certain examples, adjusting the solution pH away from the surface-neutral point of the microparticle can affect dissolution kinetics of the active agent therein. Incubation of the microparticles in the solution can be performed at or, preferably, below ambient temperature, but preferably above the freezing temperature of the solution, to minimize disintegration of the microparticles. Incubation temperature may even be lower than the freezing temperature of the solution when one or more FPDAs disclosed herein are used. For example, the incubation temperature may be between 0° C. and 15° C., between 1° C. and 10° C., between 2° C. and 5° C., and less than 5° C. In general, the concentration of the grafted polymer or charged compound in the solution for each monolayer fabrication may be equal to, less than, and/or greater than one of the following, or in a range between any two thereof: 0.05 mg/mL, 0.1 mg/mL, 0.5 mg/mL, 1 mg/mL, 10 mg/mL, 5 mg/mL, 3 mg/mL. When the microparticle core is co-incubated with the grafted polymer or charged compound in the solution, a weight ratio of the microparticle core to the grafted polymer or charged compound may be 1:1 or greater, preferably 2:1 to 10:1, more preferably 2.5:1 to 7:1.

Incubation time may be adjusted to achieve the desired charge modification (such as neutralization or charge reversal), monolayer coverage, and/or monolayer thickness. Depending on the particular reaction (such as ingredients and/or conditions), the incubation time may be equal to, shorter than, and/or longer than one of the following, or in a range between any two thereof: 10 hours, 5 hours, 3 hours, 10 minutes, 30 minutes, 100 minutes, 75 minutes, 60 minutes, 15 minutes, 5 minutes, 1 minute, 30 seconds, 10 seconds, 5 seconds, 1 second. Each monolayer may have a thickness that is equal to, less than, and/or greater than one of the following, or in a range between any two thereof: 100 nm, 50 nm, 20 nm, 5 nm, 1 nm, 0.5 nm, 0.1 nm, 2 nm, 10 nm. A typical monolayer of the present disclosure is less than 100 nm in thickness, preferably less than 10 nm.

Without wishing to be bound by any particular theory, it is believed that a factor in controlling release of the active agent from the microparticles may be the type and/or degree of interaction and/or association(e.g., non-covalent association, ionic complexation) that occurs at or near the outer surface of the microparticle core (such as the interface with the formed monolayer), which may involve the active agent, the grafted polymer and/or the charged compounds, if present, and/or other components, if any. In some cases, a strong interaction or association at this interface slows down, delays, and/or otherwise hinders dissolution of the active agent, and is believed to stabilize the surface-modified microparticle and facilitate fabrication of additional alternatingly charged monolayers, if desired. In addition, as described in greater detail below, the interaction can be further affected by the subsequent formation of additional alternatingly charged monolayers.

In another example, one or more active agents, charged and/or uncharged, may be incorporated into one or more of the monolayers through covalent and/or non-covalent associations. Such monolayer-bound active agent(s) may be the same as that of the microparticle core, or different therefrom. Such a construction may allow controlled release (e.g., extended release, sustained release) of the monolayer-bound active agent(s). Alternatively or in combination, one of more of such monolayer-bound active agent(s) may be affinity molecules, such as targeting ligands, which may selectively bring the underlying microparticle to a predetermined region to achieve targeted delivery of the active agent within the microparticle core.

In a further example, the surface-modified microparticles described above having one or more monolayers may undergo one or more physical and/or chemical treatments, preferably in a suspension, to further modify one or more characteristics of the surface-modified microparticles, such as, but not limited to, the release profile of the active agent therein. The treatments may be carried out immediately after the formation of the surface-modified microparticles and prior to any optional washing, or immediately following the optional washings. The treatment may involve manipulation of one or more parameters of the reaction mixture, such as, without limitation, temperature, pH, and/or pressure. Typically the one or more parameters may be adjusted (such as increased or decreased) from an initial value to a second value and held for a period of time, and then adjusted (such as decreased or increased) to a third value or returned or allowed to return to the initial value and held for another period of time.

The thermal treatment, for example, may involve a heating stage and a cooling stage. Prior to the additional treatment, the suspension may be kept at a relatively low temperature below ambient temperature to at least minimize dissolution of the microparticles therein, preferably the temperature at which the surface-modified microparticles are formed, more preferably 2° C. to 10° C., such as 4° C. During the heating stage, the suspension may be heated to a temperature and incubated at this elevated temperature for an incubation period of 1 minute to 5 hours, preferably 15 minutes to 1 hour, such as 30 minutes. The elevated temperature may be higher than the relatively low temperature at which the suspension was kept prior to the additional treatment, and lower than a degradation temperature of the surface-modified microparticles in the suspension, preferably between 5° C. and 40° C., more preferably between 10° C. and 30° C. The heating stage may optionally be immediately followed with a cooling stage, during which the suspension may be chilled at a temperature, rapidly or gradually in a controlled manner and optionally incubated at this depressed temperature for an incubation period of 1 minute to 5 hours, preferably 15 minutes to 1 hour, such as 30 minutes. In one example, chilling is achieved by washing with a chilled washing solution. Alternatively, the suspension may be allowed to return to or close to its original temperature or to a selected temperature below the temperature to which the suspension was heated. The depressed temperature may be lower than the elevated temperature, and higher than a freezing temperature of the suspension, preferably at or below ambient temperature, optionally equal to or different from the relatively low temperature at which the suspension was kept prior to the additional treatment, more preferably 15° C. or lower, most preferably 10° C. or lower, such as 4° C. The resulting mixture may further undergo optional washings as described herein to yield additionally treated, surface-modified, microparticles.

Surface-modified microparticles suitable for the additional treatment described above include those formed from amorphous, solid, and homogenous microparticle cores having 40% to less than 100%, or more typically 80% or greater, by weight, of an active agent as described herein. Non-limiting examples of suitable suspensions include microparticles (such as insulin microspheres) in a buffer, such as a PEG buffer containing 16% PEG, 0.7% NaCl, 67 nM Na acetate, and having a pH in the range of 5 to 8 (e.g., 5.7, 5.9, 6.5, 7.0). The microparticles may have a concentration in the buffer of 0.01 mg/ml to 50 mg/ml, preferably 0.1 mg/ml to 10 mg/ml, such as 1 mg/ml. A charged compound or a mixture of two or more thereof, such as protamine sulfate, poly-L-lysine, and/or poly-L-arginine, or a grafted polymer as described herein, may be mixed into the suspension to provide a concentration of 0.01 mg/ml to 10 mg/ml, preferably 0.1 mg/ml to 1 mg/ml, such as 0.3 mg/ml. The mixture may be incubated at the relatively low temperature, such as 4° C., and under agitation for an incubation period of 10 seconds to 5 hours, such as 1 hour, to ensure the formation of a monolayer of the grafted polymer or charged compound on the outer surface of each of the microparticle cores. Then the suspension may be subjected to the thermal treatment as described above. Optional washings may be carried out on the suspension prior to the additional treatment.

The additional treatments may be carried out immediately after the formation of any one or more of the monolayers as disclosed herein. In one example, the additional treatment may be carried out immediately after the formation of a single monolayer on the microparticle cores, the monolayer being formed of grafted polymers, positively charged compounds, or negatively charged compounds. When optionally one or more additional monolayers are formed on the first monolayer, the additional treatment may or may not be carried out immediately following the formation of such additional monolayers. In another example, two or more monolayers may be formed sequentially on the microparticle cores, and the additional treatment may be carried out only immediately after a single predetermined monolayer (such as the last monolayer; the first monolayer, or any other monolayer there between) is formed. In a further example, two or more monolayers may be formed sequentially on the microparticle cores, and the additional treatment may be carried out immediately after the formation of each and every monolayer having one or more predetermined characteristics, such as containing grafted polymers, positively charged compounds, or negatively charged compounds, or containing a particular compound (e.g., active agent, affinity molecule, derivative) or moiety (e.g., functional group, label), or being a particular monolayer from the core (e.g., first, second, third, fourth, fifth). In a further example, the additional treatment may be carried out immediately after the formation of each monolayer of a predetermined set, which may be all of the monolayers or a subset thereof.

The surface-modified microparticles following the additional treatment may display modifications in net surface charge (zeta potential) and/or release profile of the active agent therein. With certain charged compounds (such as poly-L-lysine and poly-L-arginine, but not protamine sulfate), a change (such as an increase) in the surface charge of the surface-modified microparticles may be observed. When subjected to the in vitro release protocol as disclosed herein, the additionally treated, surface-modified microparticles are capable of displaying a reduction in the 1-hour percentage of cumulative release (% CR_{1 h}) of the active agent therein, as compared to the surface-modified microparticles without the additional treatment. Inasmuch as it is believed that the initial burst of the active agent release typically occurs within the first hour, the example demonstrates that the initial burst of the active agent release may be significantly reduced by the additional treatment. The same additionally treated, surface-modified microparticles may be capable of continued, preferably sustained, release beyond 1 hour, preferably beyond 24 hours, more preferably beyond 48 hours, most preferably beyond 7 days, having a 24-hour percentage of cumulative release (% CR_{24 h}) that is greater than % CR_{1 h}. As a result of the additional treatment, the surface-modified microparticles of the present disclosure, when subjected to in vitro release in a release buffer (10 mM Tris, 0.05% Brij 35, 0.9% NaCl, pH 7.4, free of divalent cation) at 37° C., may be capable of displaying a % CR_{1 h} of 50% or less and/or a ratio of % CR_{24 h} to % CR_{1 h} of greater than 1:1. The % CR_{1 h} may preferably be 40% or less, more preferably 30% or less, further preferably 20% or less, most preferably 10% or less. The ratio of % CR₂₄ h, to % CR_{1 h} may preferably be 1.05:1 or greater, more preferably 1.1:1 or, greater, but not more than 10:1, preferably 5:1 or less, more preferably 2:1 or less, most preferably 1.5:1 or less.

Without being bound to any particular theory, it is believed that the additional treatment following the monolayer formation as disclosed herein allows the grafted polymer or charged compound in the monolayer and the molecules (e.g., the active agent, the optional carrier molecules in the microparticle core, the charged compound in the preceding monolayer) that comprises the outer surface of the substrate (e.g., the microparticle core, the preceding monolayer) to rearrange and form an association that is much stronger than the electrostatic interaction between the monolayer and the outer surface of the substrate prior to the additional treatment. It is believed that through the additional treatment a modified shell is formed on the outer surface of the surface-modified microparticle, the modified shell containing a homogenous mixture of the grafted polymer or charged compound and the molecules that form the outer surface of the substrate.

Deposition of additional alternatingly charged monolayers of charged compounds beyond the formed monolayer may further affect, among other things, the release profile of the active agent in the microparticle core. Deposition of grafted polymers also may affect, among other things, the release profile of the active agent in the microparticle core. Depending on the attractive forces at the interface between the microparticle core and the formed monolayer; strong association between the two may be observed. This may result in retarding the quantity and/or rate of release of the active agent. The release profile may be further modified by forming one or more additional alternatingly charged monolayers about the formed monolayer. Without being restricted to any particular theory, it is believed that addition of a second oppositely charged monolayer may ease the association between the formed monolayer and the microparticle core, thereby enhancing the release of the active agent. Subsequent application of the alternatingly charged monolayers, arranged consecutively with optional interleaving layers of active agents, if desired, can allow fine-tuning of active agent release from the surface-modified microparticles.

Suitable charged compounds that may be used in accordance with the present invention may be charged compounds capable of associating with any substrate, preferably by, but not limited to non-covalent association and, more preferably, electrostatic interactions. Thus, suitable charged compounds include positively charged, negatively charged, or zwitterionic, and include, but are not limited to, polyelectrolytes, charged polyaminoacids, polysaccharides, polyionic polymers, ionomers, charged peptides, charged proteinaceous compounds, charged lipids optionally in combination with uncharged lipids, charged lipid structures such as liposomes, precursors and derivatives thereof, and combinations of two or more thereof. Non-limiting examples include negatively charged polyelectrolytes such as polystyrene sulfonate (PSS) and polyacrylic acid (PAA), negatively charged polyaminoacids such as polyaspartic acid, polyglutamic acid, and alginic acid, negatively charged polysaccharides such as chondroitin sulfate and dextran sulfate, positively charged polyelectrolytes such as polyallyl amine hydrochloride (PAH) and poly(diallyldimethyl ammonium chloride (PDDA), positively charged polyaminoacids such as poly(L-lysine) hydrochloride, polyornithine hydrochloride, and polyarginine hydrochloride, and positively charged polysaccharides such as chitosan. Also useful as charged compounds in the present invention are, without limitation, biocompatible polyionic polymers (e.g., ionomers, polycationic polymers such as polycationic polyurethanes, polyethers, polyesters, polyamides; polyanionic polymers such as polyanionic polyurethanes, polyethers, polyesters, polyamides), charged proteins (e.g., protamine, protamine sulfate, xanthan gum, human serum albumin, zein, ubiquitins, and gelatins A & B), and charged lipids (e.g., phosphatidyl choline, phosphatidyl serine). Also included are derivatives (e.g., glycosylated, hyperglycosylated, PEGylated, FITC-labeled, salts thereof), conjugates, and complexes of the charged compound disclosed herein. More specifically, suitable positively charged lipids (that is, polyanionic lipids), negatively charged lipids (that is, polycationic lipids), and zwitterionic lipids include, but are not limited to 1,2-distearoyl-sn-glycero-3-phosphocholine (DSPC), 1,2-dioleoyl-sn-glycero-3-phosphoethanolamine-N-(carboxyfluorescein) (FITC-EA), 1,2-distearoyl-sn-glycero-3[phospho-rac-(1-glycerol)](sodium salt) (DSPG), 1,2-dipalmitoyl-sn-glycero-3-phosphate (monosodium salt) (DPPA), and 1,2-dioleoyl-3-dimethyl-ammonium propane (DODAP).

Furthermore, lipid structures (such as liposomes) can be used in alternate deposition with grafted polymers or charged compounds. Uncharged (such as non-ionic) lipids may be used in combination with electrically charged lipids to form one or more of the monolayers, and the molar ratio there between can be optimized to achieve minimum permeability of the active agent through the monolayer.

The surface-modified microparticle disclosed herein, typically containing a microparticle core and one or more monolayers, preferably has a release profile of the active agent that is different from that of the microparticle core. Non-limiting examples of the differences in release profile include a reduction in the initial burst, an extension of release time, a display of linear/constant release over a time period, and/or a reduction in rate of release over a prolonged time period. The surface-modified microparticles may be present, preferably in a functionally (e.g., therapeutically, pharmaceutically, diagnostically) effective amount, as a suspension or dry powder in a liquid or solid composition or formulation, in the presence or absence of one or more of preservatives, isotonicity agents, pharmaceutically acceptable carriers, and stabilizing agents. Such compositions and formulations may be administered in an effective amount to a subject for prevention or treatment of a condition or state, or as a nutritional supplement, or for the purpose of physical enhancement or psychological well-being. Such compositions and formulations may be incorporated into a diagnostic method, tool, or kit for in vitro and/or in vivo detection of a substance, condition, or disorder being present or absent, or a disposition for such a condition or disorder. For example, the substance, upon contact, may form an association (e.g., conjugate, complex) with the surface-modified microparticle or a portion thereof (such as the microparticle core), which is capable of providing one or more signals for detection. The one or more signals may be one or more moieties labeled on one or more portions of the association (e.g., the substance, the microparticles), or may be elicited upon the formation of the association (e.g., emission of light, discharge of another substance). Additionally, the surface-modified microparticles may be incorporated into a nutritional and/or dietary supplement or a food composition, or used as a food additive, for prevention and/or treatment of a condition or disorder in a subject.

The following examples are not intended to be limiting but only exemplary of specific embodiments of the invention.

### EXAMPLES

### Example 1

### Polyethylene Glycol (PEG)-Grafted Poly-L-Lysine with 6.4%,12.9%, and 19.3% Degree of Grafting

Polyethylene glycol (PEG) was grafted to fluorescein isothiocyanate (FITC)-labeled poly-L-lysine (PLL) at a 6.4%, 12.9%, and 19.3% degree of grafting. Degree of grafting is defined as the moles of PEG divided by moles of lysine (mol PEG/mol lysine). Three solutions of 25 mg/mL FITC-labeled PLL (FITC-PLL) were prepared by suspending 15 mg FITC-PLL (22 kD) (77.7 μmol lysine) in 25 mM sodium bicarbonate (pH 8.5) and vortexing the resulting suspensions. Methoxy-terminated PEG-succinimidyl α-methylbutanoate (mPEG-SMB) of molecular weight 2 kD (Nektar Therapeutics) was added to the each of the three FITC-PLL solutions and vortexed until all mPEG-SMP was dissolved. mPEG-SMP was added in an amount of 10 mg (5 μmol), 20 mg (10 μmol), or 30 mg (15 μmol) to obtain 6.4%, 12.9%, or 19.3% degree of grafting, respectively, according to the above definition). The solutions were incubated for 18 hours at room temperature with mixing provided by a platform gyratory shaker. The resulting PEG-grafted FITC-PLL was stored at 2-8° C.

### Example 2

### Insulin Microparticles Coated with a Layer of PEG-Grafted Polylysine

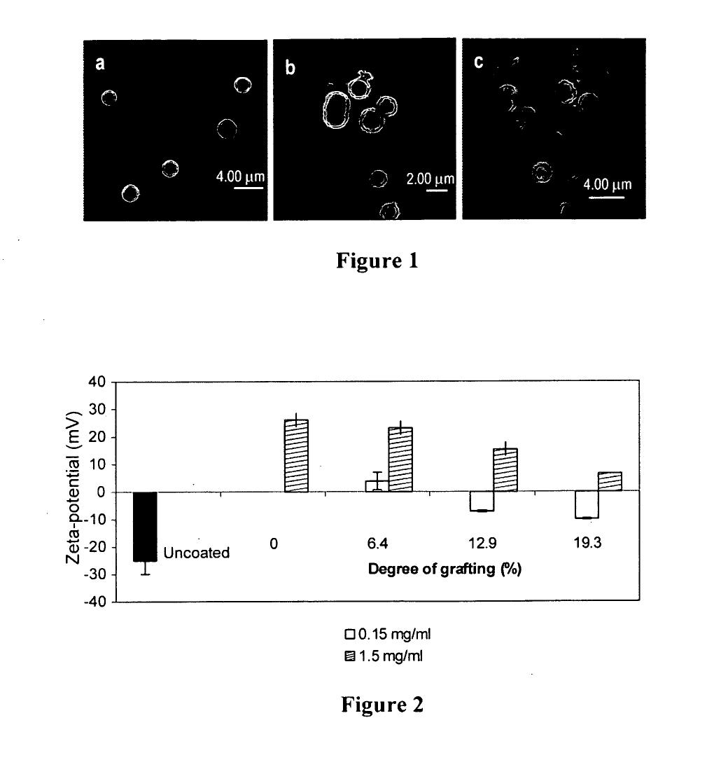

PEG-grafted FITC-labeled poly-L-lysine (PEG-grafted FITC-PLL) was used to form a layer of PEG-grafted polycations about insulin microparticle cores with a mean diameter of 1.5 micrometers. PROMAXX™ (Baxter Healthcare Corporation, Deerfield, Ill.) insulin microparticles were incubated in an aqueous solution of 16% (w/v) PEG (3.35 kD) and 0.7% (w/v) NaCl, pH 7.0 for 1 hour at 2° C. in the presence of the PEG-grafted FITC-PLL of Example 1 (0%, 6.4%, 12.9%, and 19.3% degree of grafting). PEG-grafted FITC-PLL was present in the reaction medium at 0.15 mg/mL, or 1.5 mg/mL. The FITC-PLL-coated microparticles were collected from solution by centrifugation at 3000 rpm for 15 minutes. The collected microparticless were washed twice by suspending in an aqueous solution of 16% (w/v) PEG (3.35 kD) and 0.7% (w/v) NaCl, pH 7.0, and then centrifuging at 3000 rpm for 15 minutes. The presence of the fluorescent label on the coated microparticles was observed by confocal microscopy using a Leica DMI RE2 confocal laser scanning microscope (FIG. 1). Microparticle net surface charge (zeta potential) was measured using a Zeta Potential Analyzer (Model ZetaPALS, Brookhaven Instruments Corp., Holtsville, N.Y.). The microparticle suspension was diluted in an aqueous solution of 16% (w/v) PEG, pH 7.0, and the resulting suspension was measured immediately at 8° C. Zeta-potentials of PEG-grafted FITC-PLL coated insulin microparticles are shown in FIG. 2.

### Example 3

### Polyethylene Glycol (PEG)-Grafted Poly-L-Lysine with 20% Degree of Grafting

Polyethylene glycol (PEG) was grafted to fluorescein isothiocyanate (FITC)-labeled poly-L-lysine (PLL) at a 20% degree of grafting according to the procedure of Example 1, except that PLL of 18.5 kD was used.

### Example 4

### Insulin Microparticles Coated with a Layer of Polylysine

FITC-labeled poly-L-lysine (FITC-PLL) was used to form a layer of polycations about insulin microparticle cores with a mean diameter of 1.5 micrometers. PROMAXX™ (Baxter Healthcare Corporation, Deerfield, Ill.) insulin microparticles were incubated in an aqueous solution of 16% (w/v) PEG (3.35 kD) and 0.7% (w/v) NaCl, pH 7.0 for 1 hour in the presence of FITC-PLL (18.5 kD) at 2° C. The FITC-PLL-coated microparticles were collected from solution by centrifugation at 3000 rpm for 15 minutes. The collected microparticles were washed twice by suspending in an aqueous solution of 16% (w/v) PEG (3.35 kD) and 0.7% (w/v) NaCl, pH 7.0, and then centrifuging at 3000 rpm for 15 minutes.

### Example 5

### Insulin Microparticles Coated with a Layer of PEG-Grafted Polylysine

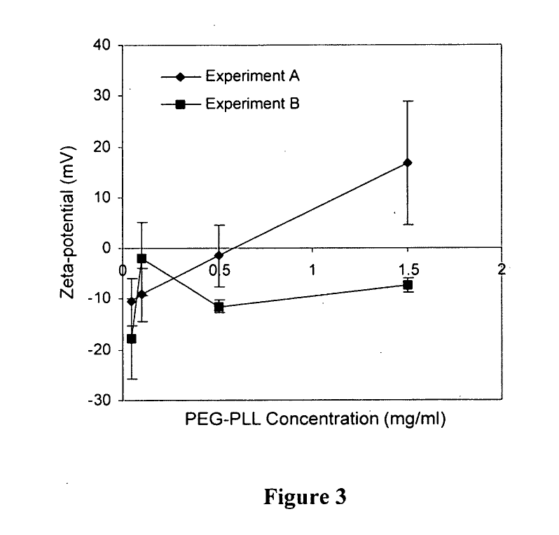

PEG-grafted FITC-labeled poly-L-lysine (PEG-grafted FITC-PLL) was used to form a layer of PEG-grafted polycations about insulin microparticle cores with a mean diameter of 1.5 micrometers. PROMAXX™ (Baxter Healthcare Corporation, Deerfield, Ill.) insulin microparticles were incubated in an aqueous solution of 16% (w/v) PEG (3.35 kD) and 0.7% (w/v) NaCI, pH 7.0 for 1 hour at 2° C. in the presence of the PEG-grafted FITC-PLL of Example 3 (20% degree of grafting). PEG-grafted FITC-PLL was present in the reaction medium at 0.05 mg/mL, 0.1 mg/mL, 0.5 mg/mL, and 1.5 mg/mL. The FITC-PLL-coated microparticles were collected from solution by centrifugation at 3000 rpm for 15 minutes. The collected microparticles were washed twice by suspending in an aqueous solution of 16% (w/v) PEG (3.35 kD) and 0.7% (w/v) NaCl, pH 7.0, and then centrifuging at 3000 rpm for 15 minutes. Microparticle net surface charge (zeta potential) was measured using a Zeta Potential Analyzer (Model ZetaPALS, Brookhaven Instruments Corp., Holtsville, N.Y.). The microparticle suspension was diluted in an aqueous solution of 16% (w/v) PEG, pH 7.0, and the resulting suspension was measured immediately at 8° C. Zeta-potentials of PEG-grafted FITC-PLL coated insulin microparticles are shown in FIG. 3.

### Example 6

### In Vitro Release of Insulin from PEG-Grafted PLL-coated Insulin Microparticles with 20% Degree of Grafting

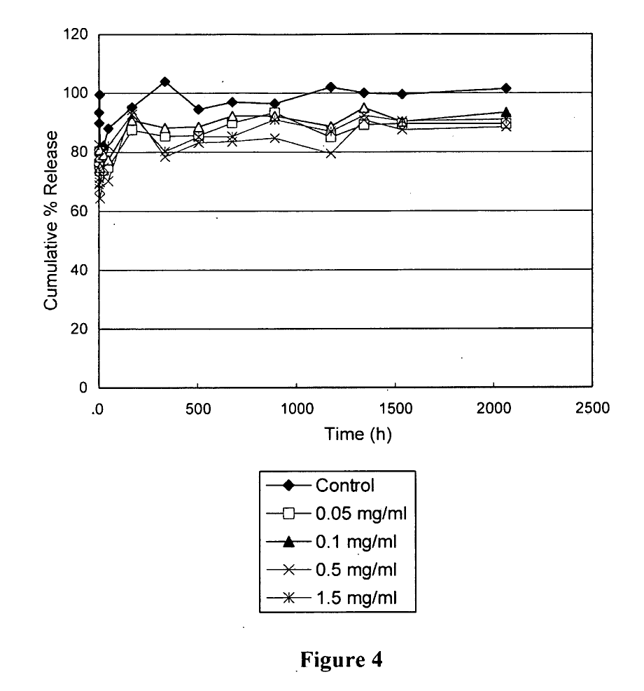

In vitro release of insulin from the PEG-grafted PLL-coated insulin microparticles of Example 5 (20% degree of grafting) was measured. A 10 mL aliquot of release medium (10 mM Tris, 0.05% Brij 35, 0.9% NaCl, pH 7.4) was added into a glass vial containing a volume of the concentrated particle suspension equivalent to 3 mg of insulin, mixed, and incubated at 37° C. At designated time intervals, 400 μL of the solution was transferred into a microcentrifuge tube, and centrifuged for 2 minutes at 13,000 rpm. A 300 μL aliquot of the supernatant was removed, and stored at −80° C. The pellet was reconstituted by addition of 300 μL of fresh release medium, and the protein content of the resulting solution was determined by bicinchoninic acid (BCA) protein assay. Total protein content of the loaded microparticles was determined by BCA assay after complete dissolution of the protein-loaded polymeric microparticles in 0.01 N HCl. Loading was defined as mass of the protein per unit mass of the microparticles. Percent of insulin released over time is shown in FIG. 4.

### Example 7

### Polyethylene Glycol (PEG)-Grafted Poly-L-Lysine with 6.4% Degree of Grafting

Polyethylene glycol (PEG) was grafted to fluorescein isothiocyanate (FITC)-labeled poly-L-lysine (PLL) at a 6.4% degree of grafting. Degree of grafting is defined as the moles of PEG divided by the moles of lysine (mol PEG/mol lysine). A solution of 25 mg/mL FITC-labeled PLL (FITC-PLL) was prepared by suspending 20 mg FITC-PLL (15-30 kD) in 25 mM sodium bicarbonate (pH 8.5) and vortexing the resulting suspension. Methoxy-terminated PEG-succinimidyl α-methylbutanoate (mPEG-SMB) of molecular weight 2 kD (Nektar Therapeutics) was added to the FITC-PLL solution and vortexed until all mPEG-SMP was dissolved. mPEG-SMP was added in an amount of 13.36 mg, giving a ratio of 1 mol PEG to 15.7 mol lysine (6.4% degree of grafting according to the above definition). The solution was incubated overnight at room temperature with mixing provided by a platform gyratory shaker. The resulting PEG-grafted FITC-PLL was stored at 4° C.

### Example 8

### Nucleic Acid Microparticles with Multiple Layers of Oppositely Charged Polyions

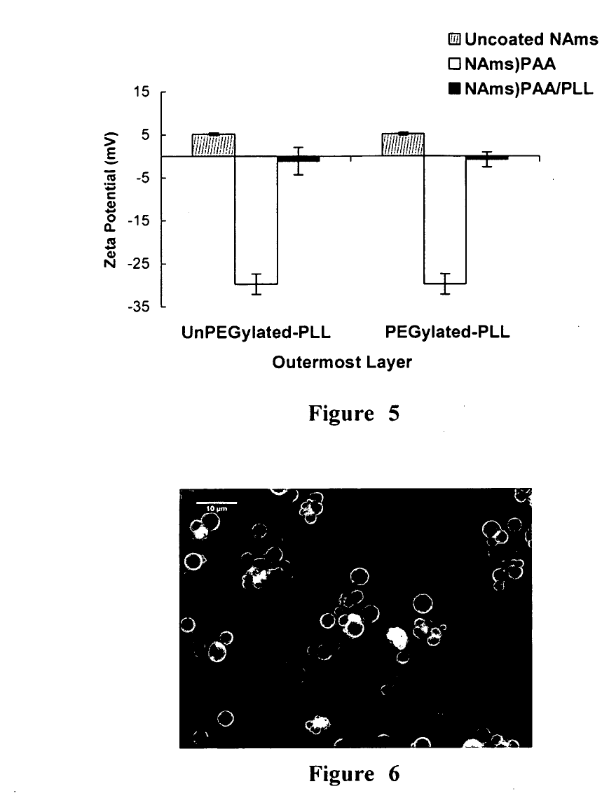

Polyaspartic acid (PAA) and FITC-labeled poly-L-lysine (FITC-PLL) were used to form multiple layers of oppositely charged polyions about nucleic acid microparticle cores. A 10 mg/mL solution of PROMAXX™ (Baxter Healthcare Corporation, Deerfield, Ill.) nucleic acid microparticles (mean diameter 2.25 μm±0.52) was prepared by suspending the microparticles in PPB (10 mM potassium phosphate buffer pH 7.0). An equal volume of a 0.3 mg/mL solution of polyaspartic acid (5-15 kD) was mixed with the microparticle suspension and then incubated for 30 minutes at 4° C. The PAA-coated microparticles were collected from solution by centrifugation at 1000 rpm for 5 minutes at 4° C. The collected microparticles were washed twice by suspending in PPB and then centrifuging at 1000 rpm for 5 minutes at 4° C. The resulting washed microparticles were suspended in PPB at 10 mg/mL. The 10 mg/mL suspension of washed PAA-coated microparticles was mixed with an equal volume of a 0.3 mg/mL solution of FITC-PLL (15-30 kD) and incubated for 1 hour at 4° C. The PAA/FITC-PLL coated microparticles were collected from solution by centrifugation at 1000 rpm for 5 minutes at 4° C. The collected microparticles were washed twice by suspending in PPB and then centrifuging at 1000 rpm for 5 minutes at 4° C. The resulting washed microparticles were suspended in PPB at 5 mg/mL. Microparticle net surface charge (zeta potential) was measured using a Zeta Potential Analyzer (Model ZetaPALS, Brookhaven Instruments Corp., Holtsville, N.Y.). The microparticle suspensions were diluted 144-fold in PPB, and the resulting suspensions were measured immediately at 6° C. Zeta-potentials of PAA-coated and PAA/FITC-PLL coated microparticles are shown in FIG. 5.

### Example 9

### Microparticles with Layers of Polyanions and PEG-Grafted Polycations

Polyaspartic acid (PAA) and PEG-grafted FITC-labeled poly-L-lysine (FITC-PLL) were used to form layers of polyanions and PEG-grafted polycations about nucleic acid microparticle cores. A 10 mg/mL solution of PROMAXX™ (Baxter Healthcare Corporation, Deerfield, Ill.) nucleic acid microparticles (mean diameter 2.25 μm±0.52) was prepared by suspending the microparticles in PPB (10 mM potassium phosphate buffer pH 7.0). An equal volume of a 0.3 mg/mL solution of polyaspartic acid (5-15 kD) was mixed with the microparticle suspension and then incubated for 30 minutes at 4° C. The polyaspartic acid (PAA)-coated microparticles were collected from solution by centrifugation at 1000 rpm for 5 minutes at 4° C. The collected microparticles were washed twice by suspending in PPB and then centrifuging at 1000 rpm for 5 minutes at 4° C. The resulting washed microparticles were suspended in PPB at 10 mg mL. The 10 mg/mL suspension of washed PAA-coated microparticles was mixed with an equal volume of a 0.3 mg/mL solution of the PEG-grafted FITC-PLL of Example 7 (6.4% degree of grafting) and incubated for 1 hour at 4° C. The PAA/PEG-grafted-FITC-PLL coated microparticles were collected from solution by centrifugation at 1000 rpm for 5 minutes at 4° C. The collected microparticles were washed twice by suspending in PPB and then centrifuging at 1000 rpm for 5 minutes at 4° C. The resulting washed microparticles were suspended in PPB at 5 mg/mL. Microparticle net surface charge (zeta potential) was measured using a Zeta Potential Analyzer (Model ZetaPALS, Brookhaven Instruments Corp., Holtsville, N.Y.). The microparticle suspensions were diluted 144-fold in PPB, and the resulting suspensions were measured immediately at 6° C. Zeta-potentials of PAA-coated and PAA/PEG-grafted-FITC-PLL coated microparticles are shown in FIG. 5. The presence of fluorescent label on the PAA/PEG-grafted-FITC-PLL coated microparticles was observed by fluorescence microscopy (FIG. 6) using a Nikon TE2000U fluorescent microscope.

### Example 10

### Uptake of Coated Microparticles by CD11b-Positive Mouse Spleen Cells

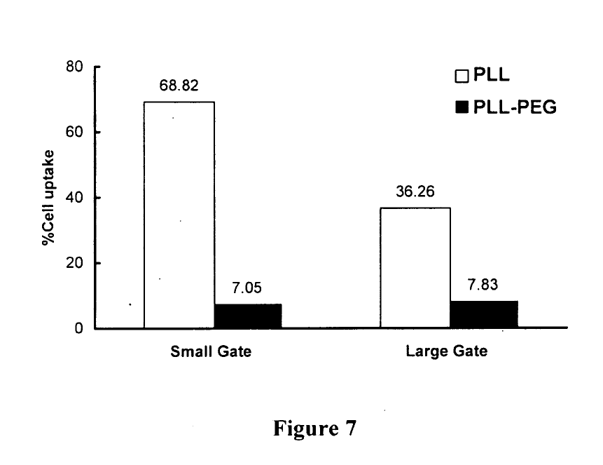

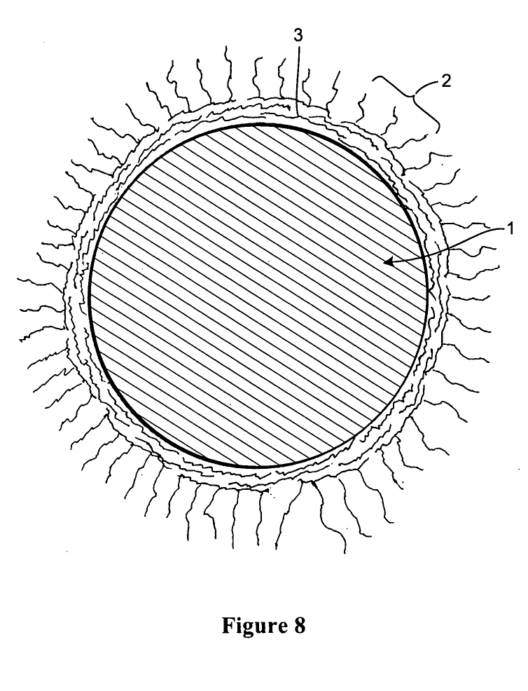

CD11b-positive mouse spleen cells were isolated and used to determine cell uptake of the coated microparticles. To obtain CD11b-positive spleen cells, mouse spleens were harvested and homogenized. The spleens were treated with red blood cell lysing buffer (HYBRI-MAX™, Sigma-Aldrich) to remove red blood cells, and the resulting suspension was centrifuged. After centrifugation, the supernatant was removed, the pellet was suspended, and the suspension was strained through a 100 μm screen. To separate CD11b-positive cells, the cell suspension was incubated with magnetic CD11b microbeads (Miltenyi Biotec) for 15 minutes, and then passed through a magnetic column. The CD11b-positive spleen cells thus obtained were suspended in RPMI media at 1.25×10⁶ cells/mL and 1 mL of the cell suspension was added to the wells of a 24-well tissue culture plate. 50 μL aliquots of the 5 mg/mL microparticle samples of Example 8 and Example 9 were added to the cell samples, and the resulting suspension was incubated for 3 hours at 37° C. at 5% CO₂. Samples were transferred from the 24-well plate to centrifuge tubes, centrifuged at 15,000 rpm for 5 minutes, and suspended in 100 mL of flow buffer (1 L phosphate-buffered saline, 10 mL 10% sodium azide, 10 mL 10% bovine serum albumin) and 4 mL of phycoerythrin-labeled CD11b antibody (BD Biosciences). After a 30 minute incubation at 4° C., the samples were centrifuged and washed to remove excess antibody, and were then suspended in 100 mL flow buffer and 100 mL 2% paraformaldehyde. The samples were then analyzed by flow cytometry to determine the extent of endocytototic uptake of the particles by CD11b-positive spleen cells. Flow cytometry analysis of the surface-modified nucleic acid microparticles was performed based on both large- and small-gated areas in order to distinguish between the endocytotic (large area) and phagocytotic (small area) uptake activities of the macrophage-resembling CD11b-positive population FIG. 7). The flow cytometry results from the large-gated analysis of cell uptake of surface-modified nucleic acid microparticles estimated uptake values of 36.3% for PEG-free-microparticles and 7.8% for PEG-grafted-microparticles. Thus, a 79% reduction in cell uptake was observed in the presence of a PEG-grafted outer-most layer on the microparticles. Analysis of cell uptake of the same samples using the small-gated area estimated uptake values of 68.8% for PEG-free-microparticles and 7.1% for PEG-grafted-microparticles. Using the small-gated area, the reduction in cell uptake was estimated at 90%.

### Example 11

### Preparation of Polyethylene Glycol (PEG)-Grafted Poly-L-Aspartic Acid

N,N′-dicyclohexylcarbodiimide (DCC) is added to a buffered solution of poly-L-aspartic acid (PAA) at a ratio of at least 1 mole DCC to 1 mole aspartic acid monomer, and the resulting suspension is vortexed. Other carboxylic acid activating reagents, such as N,N′-diisopropylcarbodiimide (DIC) and 1-ethyl-3-[3-dimethylaminopropyl]carbodiimide hydrochloride (EDC or EDAC), can be used in place of DCC. Methoxy-terminated amino-PEG (mPEG-NH2) of molecular weight 1 kD, 2 kD, 5 kD, 10 kD, or 20 kD (Nanocs, Inc.) is added to the solution of the activated PAA. The resulting mixture is vortexed until all amino PEG is dissolved, and then is incubated at room temperature for 18 hours with mixing provided by a platform gyratory shaker. The resulting PEG-grafted PAA is stored at 2-8° C., and can be used to coat microparticles as described herein.

What is claimed is:

1. A surface-modified microparticle comprising:
a microparticle core and at least one monolayer associated with the microparticle core;
wherein the monolayer comprises an amphiphilic polymer or a nonionic polymer grafted to an ionic polymer; and
the microparticle core comprises a macromolecule selected from the group consisting of carbohydrates, peptides, proteins, vectors, nucleic acids, complexes thereof, and conjugates thereof.

2. The surface-modified microparticle of claim 1, wherein the amphiphilic polymer or nonionic polymer has a degree of grafting to the ionic polymer of about 1% to about 30%.

3. The surface-modified microparticle of claim 2, wherein the degree of grafting is about 5% to about 25%.

4. The surface-modified microparticle of claim 1, wherein the amphiphilic or nonionic polymer is selected from the group consisting of polyethylene glycols, poloxamers, carbohydrate-based polymers, polyaliphatic alcohols, polyethylene glycol acrylates, poly(vinyl)polymers, polyethers, polyimides, polyesters, polyaldehydes, and copolymers, mixtures, and derivatives thereof.

5. The surface-modified microparticle of claim 1, wherein the amphiphilic or nonionic polymer is a carbohydrate-based polymer selected from the group consisting of hydroxyethyl starch polymers, polysialic acid, cyclodextrins, and mixtures thereof.

6. The surface-modified microparticle of claim 1, wherein the amphiphilic or nonionic polymer comprises polyethylene glycol and the polyethylene glycol has a molecular weight of about 500 Da to about 20,000 Da.

7. The surface-modified microparticle of claim 1, wherein the amphiphilic or nonionic polymer comprises polyethylene glycol and the polyethylene glycol has a molecular weight of about 750 Da to about 15,000 Da.

8. The surface-modified microparticle of claim 1, wherein the amphiphilic or nonionic polymer comprises polyethylene glycol and the polyethylene glycol has a molecular weight of about 900 Da to about 10,000 Da.

9. The surface-modified microparticle of claim 1, wherein the amphiphilic or nonionic polymer comprises polyethylene glycol and the polyethylene glycol has a molecular weight of about 1,000 Da to about 5,000 Da.

10. The surface-modified microparticle of claim 1, wherein the amphiphilic or nonionic polymer comprises polyethylene glycol and the polyethylene glycol has a molecular weight of about 1,500 Da to about 2,500 Da.

11. The surface-modified microparticle of claim 1, wherein the ionic polymer is selected from the group consisting of polyelectrolytes, charged polyaminoacids, charged polysaccharides, charged proteinaceous compounds, charged peptides, and mixtures thereof.

12. The surface-modified microparticle of claim 1, wherein the ionic polymer is a cationic polymer selected from the group consisting of polylysines, polyhistidines, polyornithines, polyhydroxylysines, polyarginines, polyhomoarginines, polyaminotyrosines, protamines, polydiaminobutyric acids, polyethyleneimines, polypropylenimines, polyamino(meth)acrylates, polyaminostyrenes, polyaminoethylenes, poly(aminoethyl)ethylene, polyaminoethylstyrenes, polycitrullines, diethyl amino ethyl celluloses, poly-imino tyrosines, cholestyramine-resins, poly-imino acids, 1,5-dimethyl-1,5-diazaundecamethylene polymethobromide, chitosans, poly(amidoamine) dendrimers, and mixtures and derivatives thereof.

13. The surface-modified microparticle of claim 1, wherein the ionic polymer is a cationic polymer comprising a monomer selected from the group consisting of lysine, histidine, ornithine, hydroxylysine, arginine, homoarginine, aminotyrosine, diaminobutyric acid, ethyleneimine, propylenimine, amino(meth)acrylate, aminostyrene, aminoethylene, aminoethylethylene, aminoethylstyrene, citrulline, diethyl amino ethyl glucose, imino tyrosine, (vinylbenzyl)trimethylammonium salts, imino acids, quaternary alkyl ammonium salts, amidoamines, glucosamine, and mixtures and derivatives thereof.

14. The surface-modified microparticle of claim 1, wherein the ionic polymer is a anionic polymer selected from the group consisting of polyaspartic acid, polyglutamic acid, polyacrylic acid, polymethacrylic acid, polymaleic acid, polymaleic acid monoester, heparin sulfate, dextran sulfate, polygalacturonic acid, polyalginate(polyaginic acid), polypectimic acid, polymannuronic acid, polyguluronic acid, polysialic acid, polycarboxymethyl cellulose, polyhyaluronic acid, chondroitin sulfate, chitosan sulfate, glycosaminoglycans, proteoglycans, and mixtures thereof.

15. The surface-modified microparticle of claim 1, wherein the ionic polymer is an anionic polymer comprising a monomer selected from the group consisting of aspartic acid, glutamic acid, acrylic acid, methacrylic acid, maleic acid, maleic acid monoester, heparin sulfate, dextran sulfate, galacturonic acid, alginate (aginic acid), pectimic acid, mannuronic acid, guluronic acid, sialic acid, carboxymethyl glucose, hyaluronic acid, chondroitin sulfate, sulfated glucose, sulfated glucuronic acid, sulfated iduronic acid, sulfated glucosamine, sulfated acetylgalactosamine, glycosaminoglycan-modified amino acids, sulfated carbohydrates, and mixtures thereof.

16. The surface-modified microparticle of claim 1, wherein macromolecule is selected from the group consisting of a monoclonal antibody, a polyclonal antibody, an anticancer agent, an anticoagulant, an antigen, an anti-inflammatory agent, a blood clotting factor, a cytokine, an enzyme, an enzyme cofactor, an enzyme inhibitor, a growth differentiation factor, a growth factor, an immunological agent, a parathyroid hormone, a vaccine, and mixtures thereof.

17. The surface-modified microparticle of claim 1, wherein the macromolecule is negatively charged, is positively charged, or has an inducible charge.

18. The surface-modified microparticle of claim 1, wherein the macromolecule is a nucleic acid selected from the group consisting of DNAs, RNAs, plasmids, viral vectors, oligonucleotides, antisense nucleic acids, missense nucleic acids, and a mixtures thereof.

19. The surface-modified microparticle of claim 1, wherein the microparticle core comprises an outer surface carrying a net surface charge and the monolayer associated with the microparticle core carries a net charge that is opposite in sign to the net surface charge of the outer surface.

20. The surface-modified microparticle of claim 1, further comprising a second monolayer, wherein the second monolayer is between the monolayer comprising the amphiphilic polymer or nonionic polymer grafted to the ionic polymer and an outer surface of the microparticle core.

21. The surface-modified microparticle of claim 20, wherein the monolayer comprising the amphiphilic polymer or nonionic polymer grafted to the ionic polymer is adjacent to the second monolayer.

22. The surface-modified microparticle of claim 21, wherein the second monolayer carries a net charge that is opposite in sign to the net charge of the monolayer comprising the amphiphilic polymer or nonionic polymer grafted to the ionic polymer.

23. The surface-modified microparticle of claim 1, wherein the surface-modified microparticle comprises at least first and second monolayers and the net charge of the first monolayer is opposite in sign to the net charge of the second monolayer.

24. The surface-modified microparticle of claim 23, wherein the microparticle core comprises an outer surface carrying a net surface charge, and the layer comprising the amphiphilic polymer or nonionic polymer grafted to the ionic polymer carries a net charge that is the same in sign as the net charge of the microparticle core.

25. The surface-modified microparticle of claim 1, wherein when administered to a subject, the surface-modified microparticle demonstrates at least 50% less cell uptake than a microparticle coated with a non-grafted ionic polymer.

26. The surface-modified microparticle of claim 1, wherein when administered to a subject, the surface-modified microparticle demonstrates at least 85% less cell uptake than a microparticle coated with a non-grafted ionic polymer.

27. The surface-modified microparticle of claim 1, wherein substantially no covalent bonds are present between the macromolecule and the amphiphilic polymer or nonionic polymer.

28. A pharmaceutical composition comprising a plurality of surface-modified microparticles according to claim 1.

29. The pharmaceutical composition of claim 28, wherein the surface-modified microparticles have an average size from about 0.01 μm to about 200 μm.

30. The pharmaceutical composition of claim 28, wherein the surface-modified microparticles have an average size from about 0.1 μm to about 10 μm.

31. The pharmaceutical composition of claim 28, wherein the surface-modified microparticles have an average size of from about 0.1 μm to about 5 μm.

32. The pharmaceutical composition of claim 28, wherein the surface-modified microparticles have a narrow size distribution.

33. The pharmaceutical composition of claim 32, wherein the ratio of a volume diameter of the 90th percentile of the microparticles to the volume diameter of the 10th percentile is less than or equal to about 5.

34. The pharmaceutical composition of claim 28, wherein the microparticles have a dry density of about 0.5 to about 2 g/cm³.

35. The pharmaceutical composition of claim 28, further comprising an excipient.

36. The pharmaceutical composition of claim 28, wherein said microparticles are suitable for pulmonary administration.

37. The pharmaceutical composition of claim 28, wherein said microparticles are suitable for injectable administration.

38. A process of preparing a surface-modified microparticle comprising a grafted polymer comprising:
a) providing a microparticle core comprising a macromolecule selected from the group consisting of carbohydrates, peptides, proteins, vectors, nucleic acids, complexes thereof, and conjugates thereof;
b) admixing (i) an activated amphiphilic or nonionic polymer and (ii) an ionic polymer under conditions sufficient to form a grafted polymer, said grafted polymer comprising the amphiphilic polymer or nonionic polymer grafted to the ionic polymer; and
c) admixing the grafted polymer of (b) and the microparticle core under conditions sufficient to form a surface-modified microparticle comprising a grafted polymer and having an outermost monolayer, said outermost monolayer comprising the amphiphilic polymer or nonionic polymer grafted to the ionic polymer.

39. The process of claim 38, wherein the microparticle core is provided as a surface-modified microparticle.

40. The process of claim 38, wherein the amphiphilic polymer or nonionic polymer has a degree of grafting to the ionic polymer of about 1% to about 30%.

41. The process of claim 40, wherein the degree of grafting is about 5% to about 25%.

42. The process of claim 38, wherein the amphiphilic or nonionic polymer is selected from the group consisting of polyethylene glycols, poloxamers, carbohydrate-based polymers, polyaliphatic alcohols, polyethylene glycol acrylates, poly(vinyl)polymers, polyethers, polyimides, polyesters, polyaldehydes, and copolymers, mixtures, and derivatives thereof.

43. The process of claim 38, wherein the amphiphilic or nonionic polymer is a carbohydrate-based polymer selected from the group consisting of hydroxyethyl starch polymers, polysialic acid, cyclodextrins, and mixtures thereof.

44. The process of claim 38, wherein the ionic polymer is a cationic polymer selected from the group consisting of polylysine, polyhistidine, polyornithine, polyhydroxylysine, polyarginine, polyhomoarginine, polyaminotyrosine, protamine, polydiaminobutyric acid, polyethyleneimine, polypropylenimine, polyamino(meth)acrylate, polyaminostyrene, polyaminoethylene, poly(aminoethyl)ethylene, polyaminoethylstyrene, polycitrulline, diethyl amino ethyl cellulose, poly-imino tyrosine, cholestyramine-resin, poly-imino acid, 1,5-dimethyl-1,5-diazaundecamethylene polymethobromide, chitosan, poly(amidoamine) dendrimers, and mixtures thereof.

45. The process of claim 38, wherein the ionic polymer is a cationic polymer comprising a monomer selected from the group consisting of lysine, histidine, ornithine, hydroxylysine, arginine, homoarginine, aminotyrosine, diaminobutyric acid, ethyleneimine, propylenimine, amino(meth)acrylate, aminostyrene, aminoethylene, aminoethylethylene, aminoethylstyrene, citrulline, diethyl amino ethyl glucose, imino tyrosine, (vinylbenzyl)trimethylammonium salts, imino acids, quaternary alkyl ammonium salts, amidoamines, glucosamine, and mixtures and derivatives thereof.

46. The process of claim 38, wherein the ionic polymer is a anionic polymer selected from the group consisting of polyaspartic acid, polyglutamic acid, polyacrylic acid, polymethacrylic acid, polymaleic acid, polymaleic acid monoester, heparin sulfate, dextran sulfate, polygalacturonic acid, polyalginate (polyaginic acid), polypectimic acid, polymannuronic acid, polyguluronic acid, polysialic acid, polycarboxymethyl cellulose, polyhyaluronic acid, chondroitin sulfate, chitosan sulfate, glycosaminoglycans, proteoglycans, and mixtures thereof.

47. The process of claim 38, wherein the ionic polymer is an anionic polymer comprising a monomer selected from the group consisting of aspartic acid, glutamic acid, acrylic acid, methacrylic acid, maleic acid, maleic acid monoester, heparin sulfate, dextran sulfate, galacturonic acid, alginate (aginic acid), pectimic acid, mannuronic acid, guluronic acid, sialic acid, carboxymethyl glucose, hyaluronic acid, chondroitin sulfate, sulfated glucose, sulfated glucuronic acid, sulfated iduronic acid, sulfated glucosamine, sulfated acetylgalactosamine, glycosaminoglycan-modified amino acids, sulfated carbohydrates, and mixtures thereof.

48. The process of claim 38, wherein the amphiphilic or nonionic polymer comprises polyethylene glycol and the polyethylene glycol has a molecular weight of about 500 Da to about 20,000 Da.

49. The process of claim 38, wherein the macromolecule is negatively charged, is positively charged, or has an inducible charge.

50. The process of claim 38, wherein the macromolecule is a nucleic acid selected from the group consisting of DNAs, RNAs, plasmids, viral vectors, oligonucleotides, antisense nucleic acids, missense nucleic acids, and mixtures thereof.

51. The process of claim 38, wherein the macromolecule is selected from the group consisting of a monoclonal antibody, a polyclonal antibody, an anticancer agent, an anticoagulant, an antigen, an anti-inflammatory agent, a blood clotting factor, a cytokine, an enzyme, an enzyme cofactor, an enzyme inhibitor, a growth differentiation factor, a growth factor, an immunological agent, a parathyroid hormone, a vaccine, and mixtures thereof.

52. A surface-modified microparticle comprising:
a microparticle core and at least one monolayer associated with the microparticle core;
wherein the monolayer comprises an amphiphilic polymer or a nonionic polymer grafted to an ionic polymer; and
the microparticle core comprises a macromolecule.

53. The microparticle of claim 52, wherein the macromolecule is an active agent.

54. The microparticle of claim 52, wherein the macromolecule has a molecular weight of at least 2 kD.

55. The microparticle of claim 52, wherein the macromolecule comprises a modifiable functional group.

56. The microparticle of claim 55, wherein the modifiable functional group is selected from the group consisting of an amino group, a carboxyl group, a thiol group, a hydroxyl group, an epoxy group, a haloalkyl group, an aldehyde group, a carbonyl group, an isocyanate group, an imino group, a nitrile group, and combinations thereof.

57. A process of preparing a surface-modified microparticle comprising a grafted polymer comprising:
a) providing a microparticle core comprising an active agent;
b) admixing (i) an activated amphiphilic or nonionic polymer and (ii) an ionic polymer under conditions sufficient to form a grafted polymer, said grafted polymer comprising the amphiphilic polymer or nonionic polymer grafted to the ionic polymer; and
c) admixing the grafted polymer of (b) and the microparticle core under conditions sufficient to form a surface-modified microparticle comprising a grafted polymer and having an outermost monolayer, said outermost monolayer comprising the amphiphilic polymer or nonionic polymer grafted to the ionic polymer.

58. The process of claim 57, wherein the active agent comprises a small molecule having a molecular weight less than 2 kD.

59. The process of claim 57, wherein the active agent comprises a macromolecule having a molecular weight of at least 2 kD.

60. The process of claim 57, wherein the active agent comprises a modifiable functional group.

61. The process of claim 60, wherein the modifiable functional group is selected from the group consisting of an amino group, a carboxyl group, a thiol group, a hydroxyl group, an epoxy group, a haloalkyl group, an aldehyde group, a carbonyl group, an isocyanate group, an imino group, a nitrile group, and combinations thereof.
